(19) United States
(12) Patent Application Publication
(10) Pub. No.: US 2026/0092316 A1
(43) Pub. Date: Apr. 2, 2026

(54) Spatially Resolved Cellular Profiling

(71) Applicant: TTP PLC, Melbourn (GB)

(72) Inventors: Soma Turi, Melbourn (GB); Lauren Victoria Elizabeth Laing, Melbourn (GB); Giles Hugo William Sanders, Melbourn (GB); Michael Ian Walker, Melbourn (GB); Richard Janse Van Rensburg, Melbourn (GB); Yuhang Xie, Melbourn (GB); Niall Keating, Melbourn (GB)

(21) Appl. No.: 19/112,915

(22) PCT Filed: Sep. 20, 2023
(86) PCT No.: PCT/GB2023/052430

§ 371 Date: Mar. 18, 2025

(30) Foreign Application Priority Data
Sep. 20, 2022 (GB) 2213732.7
Apr. 19, 2023 (GB) 2305769.8

(51) Int. Cl. C12Q 1/6841; G16B 45/00

(52) U.S. Cl. CPC C12Q 1/6841; G16B 45/00

(57) ABSTRACT

There is described a method of spatially resolved cellular profiling for integrating profiling data of a cell or cell-derived material with spatial positioning of the cell or cell-derived material, the method comprising: contacting the surface of a cell or tissue sample with a population of particles, wherein the particles comprise at least 3 distinguishable subpopulations, wherein each of the at least 3 distinguishable subpopulations has a distinguishable trait that can be determined by imaging, and wherein the particles comprise binding molecules that bind to target biomolecules from the sample; imaging the sample and the population of particles; profiling the particles to generate profiling data corresponding to each particle; and providing a virtual map of the spatially resolved profiling data with respect to the sample image.

### FIELD OF THE INVENTION

The present invention relates to a method of spatially resolved cellular profiling for integrating profiling data of a cell or cell-derived material with spatial positioning of the cell or cell-derived material.

### BACKGROUND TO THE INVENTION

Spatial multi-omics has the potential to reveal panoramas of gene and protein expression, enabling better biomarker discovery and facilitating drug development.

Current techniques of spatial profiling by sequencing are based on the use of DNA barcodes printed onto and conjugated to a functionalised slide. RNA transcripts from the tissue section are captured on the microscope slide seeded with a “lawn” of conjugated capture probes, similar to a microarray. The coordinates of captured molecules each possess a unique spatial barcode, which is integrated into the library molecules during first strand synthesis, enabling the user to later bioinformatically reassign reads to the spatial coordinates from which they came.

Printing-based technologies such as those used in spatial biology present both sample coverage and resolution challenges. Typically, printed DNA barcodes sizes only offer 5-10 cell resolution per spatial coordinate. To prevent spatial barcode islands from merging and hindering data analysis, spots are printed in a well spaced array. The gap in coverage and low resolution of current technologies leaves a significant fraction of the tissue section unanalysed (up to 70%) and requires a large number of different unique barcodes to be printed, which comes with high consumable costs and cumbersome manufacturing processes.

In particular, 10× Genomics Visium spatial technology uses an array of capture spots with a spot size of 55 μm and a 100 μm centre to centre pitch. The 55 μm spot size is larger than the size of many mammalian cells (average 10-20 μm in diameter), and as such this technique does not offer single cell resolution as there are ˜10 cells per capture spot. This technology provides relatively low sensitivity, and is a high cost, labour intensive process, with a limited capacity to do protein profiling.

An aim of the present invention is to provide a method for single-cell resolution spatial omics that overcomes or mitigates one or more of the problems associated with the prior art technologies.

### SUMMARY OF THE INVENTION

There is provided a method of spatially resolved cellular profiling for integrating profiling data of a cell or cell-derived material with spatial positioning of the cell or cell-derived material. The method comprises:
(a) placing a cell or tissue sample onto a sample-receiving surface of a substrate;
(b) contacting the surface of the sample with a population of particles, wherein the particles comprise at least 3 distinguishable subpopulations (preferably, at least 5 distinguishable subpopulations), wherein each of the at least 3 distinguishable subpopulations (preferably, at least 5 distinguishable subpopulations) has a distinguishable trait that can be determined by imaging, and wherein the particles comprise binding molecules that bind to target biomolecules from the sample;
(c) imaging the sample to provide a sample image;
(d) imaging the population of particles to provide a particle image, wherein the distinguishable trait and spatial positioning of the particles of the at least 3 distinguishable subpopulations (preferably, at least 5 distinguishable subpopulations) can be determined relative the surface of the sample;
(e) capturing target biomolecules from the sample to the binding molecules, such that target biomolecules that are in close proximity to a particle bind to that particle;
(f) removing the population of particles from the surface of the sample;
(g) profiling the particles to generate profiling data corresponding to each particle, wherein the profiling step comprises profiling the target biomolecules bound to the particles;
(h) calculating similarity scores for pairs or pluralities of particles based on the profiling data;
(i) assigning the profiling data corresponding to a particle to a spatial position of a particle in the particle image, based on the similarity scores and/or the particle image;
(j) providing a virtual map of the spatially resolved profiling data with respect to the sample image.

The method of the invention is a spatial omics method that allows profiling information associated with a sample to be linked back to the spatial information of the sample at single cell or subcellular resolution. The method of the invention overcomes the limitation of the large number of printed barcodes required in existing spatial technologies of the prior art. The method works with a significantly reduced number of printed barcodes, or no printed barcodes at all. Accordingly, the method of the invention allows for high efficiency, high coverage single cell or subcellular resolution spatial multi-omics that is not reliant on a pre-selected panel of targets and can therefore be used both in true discovery and the fundamental research space.

The term “spatially resolved cellular profiling for integrating profiling data of a cell or cell-derived material with spatial positioning of the cell or cell-derived material” is intended to mean characterising the biological material within a cell or tissue sample (e.g., characterising the transcriptome, epigenome, lipidome, metabolome or proteome) to generate profiling data, while preserving spatial information relating to the origin of the biological material within the cell or tissue sample. The method of the invention provides the advantage that profiling data can be mapped/assigned back to a specific spatial location within a cell or tissue sample based on neighbourhood similarities.

### Step (a)

The cell or tissue sample may be any cell or tissue, including intact cells and tissues, non-intact cells and tissues, and materials derived from cells and tissues (e.g., cell lysates, tissue lysates). For example, the “cell-derived material” may be a cell or tissue lysate originating from a cell or tissue sample. In some embodiments, the cell or tissue sample is a cytology sample (e.g., a cytospin sample, a smear, an aspirate, a tissue scrape or swab), or a tissue slice (e.g., fresh, frozen, or fixed). Typically, the cell or tissue sample is a tissue slice. The cell or tissue sample may be prepared in any suitable manner. When a tissue slice is used, the slice may be fixed (e.g., in formalin, paraformaldehyde, osmium tetroxide, etc., or by snap-freezing) and/or embedded (e.g., in paraffin, OCT, carbowax, methacrylate, epoxy resin, agar, celloidin media, gelatin, low-met agarose etc.). The tissue slice may be cut using any suitable means, for example using a microtome, vibratome or compresstome. Typically, the tissue slice is <10 μm thick. Typically, the tissue slice is 5-10 μm thick. The tissue may be sliced at any suitable temperature (e.g., room temperature, −40 to −60° C., −140 to −160° C.). In some embodiments, the cell or tissue sample is a formalin-fixed paraffin embedded tissue slice. In other embodiments, the cell or tissue sample is a cryo-frozen OCT-embedded tissue slice.

In some embodiments, the sample is 5-500 mm². In some embodiments, the sample is 10-400 mm². In some embodiments, the sample is 20-300 mm². In some embodiments, the sample is 30-200 mm². In some embodiments, the sample is 40-100 mm². In various embodiments, the sample is at least 30 mm².

In step (a), the cell or tissue sample is placed onto a sample-receiving surface of a substrate. The term “placing” is understood to mean positioning the sample onto the sample-receiving surface by any suitable means. For example, the sample (e.g., a tissue slice) may be overlaid onto the sample-receiving surface. In another example, the sample (e.g., a fluid cytology sample) is smeared across the sample-receiving surface. In another example, the sample (e.g., a fluid cytology sample) is flowed across the sample-receiving surface.

The substrate may be any suitable surface for receiving the cell or tissue sample. The sample-receiving surface of the substrate is the surface of the substrate which contacts the cell or tissue sample. In various embodiments, the substrate is a microscope slide, a chip, a solid array, or a coverslip. In some embodiments, the substrate is a microscope slide.

### Step (b)

In step (b) of the method, the surface of the sample is contacted with a population of particles. The population of particles may be any suitable population of particles that allows single cell or subcellular spatial resolution. Typically, the average particle diameter is matched to the average cell diameter of the cells within the cell or tissue sample. Typically, the number of particles in the population of particles is approximately the same as the number of cells within the cell or tissue sample. In some embodiments, the number of particles in the population of particles is the same as the number of cells within the cell or tissue sample±20%. In some embodiments, the number of particles in the population of particles is the same as the number of cells within the cell or tissue sample±10%. In some embodiments, the number of particles in the population of particles is the same as the number of cells within the cell or tissue sample±5%.

The population of particles may be made from any suitable material, for example silica, polymer (e.g., PS, PMMA, PE, PET, PP, PLGA) or resin (e.g., urea-formaldehyde, phenol-formaldehyde). In some embodiments, the population of particles is a population of microbeads, a population of microspheres, a population of nanobeads, or a population of DNA origami constructs. Typically, the population of particles is a population of microbeads. Typically, the population of particles is a population of microbeads made from silica, polymer or resin.

In some embodiments, the particles are coated for surface optimisation. For example, the particles may be coated with polyethylene, hydrogel or silane. In various embodiments, the particles comprise surface modifications, such as carboxylates, sulphates, aldehydes, amines or NHS esters.

The average diameter of the particles or microbeads may range from 1 to 50 μm. In some embodiments, the average diameter of the particles or microbeads is 2-45 μm. In some embodiments, the average diameter of the particles or microbeads is 3-40 μm. In some embodiments, the average diameter of the particles or microbeads is 4-35 μm. In some embodiments, the average diameter of the particles or microbeads is 5-30 μm. In some embodiments, the average diameter of the particles or microbeads is 6-25 μm. In some embodiments, the average diameter of the particles or microbeads is 7-20 μm. In some embodiments, the average diameter of the particles or microbeads is 8-18 μm. In some embodiments, the average diameter of the particles or microbeads is 9-16 μm. In some embodiments, the average diameter of the particles or microbeads is 10-15 μm. Typically, the average diameter of the particles or microbeads is 5 to 15 μm. More typically, the average diameter of the particles or microbeads is 10 μm. Matching the diameter of the particles to the average cell diameter of the cell or tissue sample has the advantage of providing single cell resolution. This is a significant improvement over the techniques of the prior art that use patches of printed barcodes, as the printing of these patches is limited by printing technologies to around 20-30 μm diameters. Accordingly, these techniques in the prior art do not provide true single cell resolution, in contrast to the present invention.

The standard deviation of the average diameter may be ±20%. The standard deviation of the average diameter may be ±15%. The standard deviation of the average diameter may be ±10%. The standard deviation of the average diameter may be ±5%. The standard deviation of the average diameter may be ±2%. The standard deviation of the average diameter may be ±1%.

In various embodiments, the average diameter of the particles or microbeads is 5-15 μm±20%. In various embodiments, the average diameter of the particles or microbeads is 8-12 μm±20%.

In various embodiments, the population of particles comprises two or more subpopulations of particles with each of the two or more subpopulations having a different average diameter. The description relating to the average diameter of the population of particles or microbeads is equally applicable to the average diameter of the subpopulations of particles. For example, the population of particles may comprise a first subpopulation of particles with a first average diameter (e.g., 10 μm) and a second subpopulation of particles with a second average diameter (e.g., 20 μm), wherein the first and second average diameters are different.

The step of contacting the surface of the sample with a population of particles can be achieved in any suitable manner which deposits the particles on the surface of the sample randomly and with high efficiency, and with a spacing approximating the cell spacing of the cell or tissue sample. In certain embodiments, sensitivity can be improved by decreasing the spacing such that there are multiple particles per cell.

The term “contacting” encompasses embodiments where there is a full or partial membrane or other structure between the surface of the sample and the population of particles which allows target biomolecules from the sample to bind to the binding molecules on the particles. For example, there may be a biomolecule-permeable membrane positioned between the population of particles and the sample.

In some embodiments, the step of contacting the surface of the sample with the population of particles involves applying a solution comprising the population of particles to the surface of the sample. The solution may be flowed over the surface of the sample in one or more directions. The solution may be bubbled on the surface of the sample (i.e., held in place by surface tension). The substrate may be a fluid container and the solution may be applied to the surface of the sample and retained on the surface of the sample by the fluid container. The solution may be spin-coated or vibrational-coated over the surface of the sample. The solution may be printed or sprayed onto the surface of the sample. All of these embodiments provide the advantage that the population of particles is randomly applied to the surface of the sample.

In some embodiments, the population of particles is fixed to or trapped in a particle-receiving surface of a particle holder substrate (may also be referred to as a particle holder, or a particle substrate). In these embodiments, the step of contacting the surface of the sample with the population of particles involves overlaying the sample with the particle-receiving surface of the particle holder substrate. For example, the sample is positioned on the sample-receiving surface of a substrate, and the particles are positioned on the particle-receiving surface of a particle holder substrate; to contact the sample with the population of particles, the sample-receiving surface (holding the sample) and the particle-receiving surface (holding the particles) are overlaid, i.e., sandwiched together.

The population of particles may be trapped within cavities or wells in the particle-receiving surface of the particle holder substrate. For example, the cavities or wells may be formed by laser drilling. The population of particles may be fixed to the particle-receiving surface of the particle holder substrate by surface chemistry. In some embodiments, the population of particles is fixed by surface chemistry within cavities or wells in the particle-receiving surface of the particle holder substrate. In various embodiments, the population of particles is fixed to the particle-receiving surface of the particle holder substrate by affinity trapping using non-covalent interactions (e.g., streptavidin-biotin interactions). In particular embodiments, the population of particles is fixed to the particle-receiving surface of the particle holder substrate by covalent or electrostatic interactions (e.g., silica-polylysine interactions, UV catalysed covalent linkages, amine with N-hydrosuccimide interactions, etc). In certain embodiments, the population of particles is affixed to the particle-receiving surface of the particle holder substrate using magnetism. For example, the population of particles may be magnetic (e.g., paramagnetic particles or beads) and affixed to the particle-receiving surface of the particle holder substrate through the particle-receiving surface being a magnet (e.g., permanent or induced).

When a particle holder substrate is used, the population of particles may be deposited to the particle-receiving surface in any suitable manner which deposits the particles randomly and with high efficiency, and with a spacing approximating the cell spacing of the cell or tissue sample. In certain embodiments, sensitivity can be improved by decreasing the spacing such that there are multiple particles per cell.

When a particle holder substrate is used, some embodiments involve the use of an intermediate substrate that guides/aligns the sample-receiving surface of a substrate with the particle-receiving surface of the particle holder substrate.

In preferable embodiments, the population of particles is randomly distributed on the particle holder substrate. For example, the particles are not purposefully positioned at specific coordinates of the particle holder substrate. This provides the advantage that the particle holder substrate is cheaper and easier to manufacture compared to existing technologies where lawns of barcodes are printed onto slides in a non-random manner. In various embodiments, 70% of the population is randomly distributed on the particle holder substrate. In various embodiments, 80% of the population of particles is randomly distributed on the particle holder substrate. In various embodiments, 90% of the population of particles is randomly distributed on the particle holder substrate. In various embodiments, 95% of the population of particles is randomly distributed on the particle holder substrate. In various embodiments, 98% of the population of particles is randomly distributed on the particle holder substrate. In various embodiments, 99% of the population of particles is randomly distributed on the particle holder substrate. In various embodiments, 100% of the population of particles is randomly distributed on the particle holder substrate.

In some embodiments, the population of particles is applied to the sample-receiving surface of a substrate before the sample has been placed onto the sample-receiving surface of a substrate. In these embodiments, the sample is subsequently placed onto the sample-receiving surface of the substrate which comprises the population of particles, thus achieving the contact between the surface of the sample with the population of particles. The population of particles may be applied to the sample-receiving surface of the substrate in any manner, including the embodiments as described above for contacting the surface of the sample with a population of particles (applying as a solution, flowing, bubbling, applying to a fluid container, fixation (e.g., by surface chemistry), trapping (e.g., within cavities or wells), etc.).

In various embodiments, the particles are overlaid on the surface of the sample. In other embodiments, the sample is overlaid on the population of particles.

The target biomolecules from the sample may be any suitable biomolecules of interest to be profiled that originate from the sample (i.e., the cell or tissue sample). In some embodiments, the target biomolecules are DNA molecules, RNA molecules, proteins (e.g., proteins and/or protein aggregates/oligomers), lipids, peptides, or epigenetic marks of DNA, RNA or histones. In various embodiments, the target biomolecules are DNA molecules, RNA molecules, peptides or proteins. In various embodiments, the target biomolecules are DNA molecules or RNA molecules. In many embodiments, the target biomolecules are RNA molecules. In particular embodiments, the RNA molecules are mRNA molecules, tRNA molecules, siRNA molecules, rRNA molecules, snRNA molecules, miRNA molecules, aRNA molecules, tmRNA molecules, snoRNA molecules, piRNA molecules, and/or lncRNA molecules. In certain embodiments, the RNA molecules are mRNA molecules.

In some embodiments, the target biomolecules are RNA molecules, peptides or proteins and the profiling step is done by RNA sequencing or mass spectrometry. In various embodiments, the target biomolecules are RNA molecules and the profiling step is done by RNA sequencing. In some embodiments, the target biomolecules are peptides or proteins and the profiling step is done by mass spectrometry.

The particles comprise binding molecules that bind to target biomolecules from the sample. Typically, each particle of the population of particles comprises binding molecules that bind to target biomolecules from the sample. The binding molecules may immobilise the target biomolecules to the surface of the particles. The binding molecules may be any suitable binding molecules for binding the target biomolecules. For example, the binding molecules may bind to the target biomolecules by hybridisation, conjugation, ligation, affinity, etc. In some embodiments, the binding molecules are polyT oligonucleotides. This provides the advantage that the polyT oligonucleotide binds to an mRNA target molecule and immobilises the mRNA target molecule to the surface of the particle. In various embodiments, the binding molecules are antibodies or antibody fragments. This provides the advantage that the antibody or antibody fragment binds to a peptide or protein target molecule and immobilises the target molecule to the surface of the particle. In some embodiments, the binding molecules are random hexamers (e.g. polyN). In various embodiments, the binding molecules are RNA aptamers. This provides the advantage that the RNA aptamer binds to an RNA target molecule and immobilises the RNA target molecule to the surface of the particle.

Each distinguishable subpopulation has a distinguishable trait that can be determined by imaging. In other words, each distinguishable subpopulation is different in some manner (e.g., by a visual barcode, in fluorescent tag colour, fluorescent tag intensity, particle size, particle refractive index, particle shape magnetic properties, and combinations thereof, such as colour and size, etc.) that allows each distinguishable subpopulation to be identified and distinguished from each other subpopulation when imaged. The distinguishable trait may be a single trait (or characteristic), e.g., the distinguishable subpopulations are all a different colour, such as blue, red, green, yellow, purple, etc, or have a different visual barcode. Alternatively, the distinguishable trait (or characteristic) may be a combination of two or more traits. For example, the distinguishable trait may be a combination of fluorescent tag colour and particle size.

By way of example only, there may be 9 subpopulations: small red microbeads, medium red microbeads, large red microbeads, small blue microbeads, medium blue microbeads, large blue microbeads, small green microbeads, medium green microbeads, and large green microbeads. Each of the 9 subpopulations can be distinguished from each other by imaging by determining the microbead colour (red, blue or green) and the microbead size (small, medium or large).

As a further example, there may be 25 subpopulations by using 5 different sizes of microbeads and 5 different fluorescent tag colours.

As a further example, there may be 27 subpopulations by using 3 different sizes of microbeads, 3 different fluorescent tag colours (e.g., using quantum dots) and 3 different fluorescent tag intensities e.g., using quantum dots).

As a further example, there may be 5 particles with different visual barcodes and a larger number of non-barcoded particles; each of the barcoded particles forms a distinguishable subpopulation and the non-barcoded particles form a non-distinguishable subpopulation.

In embodiments where fluorescent tag colour is used as a distinguishable trait, the fluorescent tag may be a fluorescent antibody, a fluorochrome, a quantum dot, a fluorescently labelled DNA origami construct, a fluorescently labelled RNA origami construct, or fluorescently labelled DNA or RNA (e.g., using fluorescent nucleoside analogs). The fluorescent tag may be conjugated to the surface of the particle or dissolved in the body of the particle (e.g., dissolved in the body of a polymer/resin microbead).

The distinguishable trait may be a combination of two to five traits. The distinguishable trait may be a combination of two traits. The distinguishable trait may be a combination of three traits. The distinguishable trait may be a combination of four traits. The distinguishable trait may be a combination of five traits. In some embodiments, the distinguishable trait is selected from visual barcode, fluorescent tag colour, fluorescent tag intensity, particle size, particle refractive index, particle shape, magnetic property, or a combination thereof. In some embodiments, the distinguishable trait is selected from visual barcode, fluorescent tag colour, fluorescent tag intensity, particle size, or a combination thereof. Using a combination of distinguishable traits provides the advantage that the imaging set up is cheaper and requires fewer optical filters.

The number of distinguishable subpopulations is at least 3 subpopulations. In some embodiments, the at least 3 distinguishable subpopulations is at least 5 subpopulations. In some embodiments, the at least 3 distinguishable subpopulations comprises 3-100 distinguishable subpopulations. In some embodiments, the at least 3 distinguishable subpopulations comprises 5-100 distinguishable subpopulations. In some embodiments, the at least 3 distinguishable subpopulations comprises 10-90 distinguishable subpopulations. In some embodiments, the at least 3 distinguishable subpopulations comprises 20-80 distinguishable subpopulations. In some embodiments, the at least 3 distinguishable subpopulations comprises 30-70 distinguishable subpopulations. In some embodiments, the at least 3 distinguishable subpopulations comprises 40-60 distinguishable subpopulations. In some embodiments, the at least 3 distinguishable subpopulations comprises 5-50 distinguishable subpopulations. In some embodiments, the at least 3 distinguishable subpopulations comprises 50-100 distinguishable subpopulations. In some embodiments, the at least 3 distinguishable subpopulations comprises at least 10 distinguishable subpopulations. In some embodiments, the at least 3 distinguishable subpopulations comprises at least 20 distinguishable subpopulations. In some embodiments, the at least 3 distinguishable subpopulations comprises at least 30 distinguishable subpopulations.

In some embodiments, the population of particles comprises fewer than 100 distinguishable subpopulations having a distinguishable trait that can be determined by imaging. In some embodiments, the population of particles comprises fewer than 50 distinguishable subpopulations having a distinguishable trait that can be determined by imaging. In some embodiments, the population of particles comprises fewer than 40 distinguishable subpopulations having a distinguishable trait that can be determined by imaging. In some embodiments, the population of particles comprises fewer than 30 distinguishable subpopulations having a distinguishable trait that can be determined by imaging. In some embodiments, the population of particles comprises fewer than 20 distinguishable subpopulations having a distinguishable trait that can be determined by imaging. In some embodiments, the population of particles comprises fewer than 10 distinguishable subpopulations having a distinguishable trait that can be determined by imaging.

The skilled person will recognise that, typically, the number of particles scales with the area of the sample. Accordingly, the skilled person will be able to select a suitable number of particles that is appropriate for their sample (as well as a suitable number of distinguishable subpopulations and number of particles within each subpopulation).

In some embodiments, each distinguishable subpopulation consists of 1-1000 particles. In some embodiments, each distinguishable subpopulation consists of 1-900 particles. In some embodiments, each distinguishable subpopulation consists of 1-800 particles. In some embodiments, each distinguishable subpopulation consists of 1-700 particles. In some embodiments, each distinguishable subpopulation consists of 1-600 particles. In some embodiments, each distinguishable subpopulation consists of 1-500 particles. In some embodiments, each distinguishable subpopulation consists of 1-400 particles. In some embodiments, each distinguishable subpopulation consists of 1-300 particles. In some embodiments, each distinguishable subpopulation consists of 1-200 particles. In some embodiments, each distinguishable subpopulation consists of 1-100 particles. In some embodiments, each distinguishable subpopulation consists of 2-90 particles. In some embodiments, each distinguishable subpopulation consists of 5-80 particles. In some embodiments, each distinguishable subpopulation consists of 10-70 particles. In some embodiments, each distinguishable subpopulation consists of 20-60 particles. In some embodiments, each distinguishable subpopulation consists of 30-50 particles.

The skilled person will appreciate that in some embodiments, all of the beads in the population of particles will fall into one of the distinguishable subpopulations (e.g., into one of the at least 3 distinguishable subpopulations). By way of example only, there may be 1000 particles in total, with 200 particles of a first colour, 200 particles of a second colour, 200 particles of a third colour, 200 particles of a fourth colour, and 200 particles of a fifth colour. As a further example, there may be 900 particles in total, with 300 particles of a first colour, 300 particles of a second colour, and 300 particles of a third colour. The skilled person will further appreciate that in other embodiments, there may be a non-distinguishable subpopulation of particles that do not have a distinguishable trait that can be determined by imaging. By way of example only, there may be 1000 particles in total, with 20 particles of a first colour, 20 particles of a second colour, 20 particles of a third colour, 20 particles of a fourth colour, 20 particles of a fifth colour, and 900 unlabelled particles. As a further example, there may be 900 particles in total, with 30 particles of a first colour, 30 particles of a second colour, 30 particles of a third colour, and 810 unlabelled particles.

In some embodiments, 100% of the particles have a distinguishable trait (i.e., fall into one of the distinguishable subpopulations). In some embodiments, 10-90% of the particles have a distinguishable trait. In some embodiments, 15-85% of the particles have a distinguishable trait. In some embodiments, 20-80% of the particles have a distinguishable trait. In some embodiments, 25-75% of the particles have a distinguishable trait. In some embodiments, 30-70% of the particles have a distinguishable trait. In some embodiments, 35-65% of the particles have a distinguishable trait. In some embodiments, 40-60% of the particles have a distinguishable trait. In some embodiments, 45-55% of the particles have a distinguishable trait. In some embodiments, 5-50% of the particles have a distinguishable trait. Typically, 10% of the particles have a distinguishable trait.

### Steps (c) and (d)

The method involves imaging the sample to provide a sample image, and imaging the population of particles to provide a particle image. The sample may be imaged using any suitable imaging technique. In some embodiments, the sample is imaged by bright field microscopy or by fluorescent microscopy. In some embodiments, multiple slices of the same tissue sample are used and the imaging step involves imaging each tissue slice.

In various embodiments, the sample is stained using a suitable technique, prior to imaging the sample. Various routine stains are known to one skilled in the art. For example, the sample may be stained using histology stains (e.g., H&E, Masson Triple, Aldehyde Fuchsin, Weigert's, Verhoeff, Silver, Periodic Acid Schiff, Acridine orange, Carmine, Coomassie blue, DAPI, Hoechst, Methylene blue, Nile blue, Nile red, etc.) or immunohistochemistry stains (e.g., using chromogenic or fluorescent antibodies). In preferable embodiments, the sample is stained with a fluorescent stain, such as Hoechst stain and imaged using fluorescent microscopy. Prior sample staining provides the advantage that the cells within the tissue can be identified (e.g., based on cell type or phenotype, such as biomarker presence). In some embodiments, the sample is stained and imaged before being contacted by the population of particles. This provides the advantage that there is no interference between the signal from the sample and the signal from the particles.

The population of particles may be imaged using any suitable imaging technique that can determine the distinguishable trait of each particle of the distinguishable subpopulations (i.e., fluorescent tag colour, size, etc) and the spatial positioning of those particles. In some embodiments, the population of particles is imaged by fluorescence microscopy (e.g., hyperspectral, confocal or standard), surface electron microscopy, mirror electron microscopy, quantitative phase imaging, x-ray imaging, or electron beam imaging. The term “imaging” may also refer to any other detection method or combination of detection methods that can determine the distinguishable trait of each particle of the distinguishable subpopulations and the spatial positioning of those particles, for example, measuring magnetic forces. In some embodiments, the population of particles is imaged by fluorescence microscopy and bright-field microscopy. In preferable embodiments, the population of particles is imaged by fluorescence microscopy. The imaging technique is chosen based on the distinguishing traits of the subpopulations of particles. For example, if the distinguishing trait is fluorescent tag colour or visual barcodes, the imaging technique will be fluorescent microscopy.

In some embodiments, only the distinguishable subpopulations are imaged (allowing the determination of the distinguishable trait and spatial position of each particle of the distinguishable subpopulations). In other embodiments, all of the particles are imaged (allowing the determination of the distinguishable trait of each particle of the distinguishable subpopulations, and allowing the determination of the spatial position of all of the particles). The skilled person will recognise that the term “imaging the population of particles to provide a particle image” may refer to imaging all of the particles in the population of particles, but may alternatively refer to imaging the particles of the distinguishable subpopulations (and not imaging the particles of any non-distinguishable subpopulation).

In some embodiments, the steps of imaging the sample and imaging the population of particles are done simultaneously. For example, steps (c) and (d) of the method may be combined into a single step of imaging the sample (contacted by the population of particles) to provide an image of the population of particles with respect to the sample, wherein the distinguishing trait and spatial positioning of the particles can be determined relative the surface of the sample. The imaging step may comprise taking multiple images at different focal distances (e.g., a first focal distance to image the sample, and a second focal distance to image the particles). In various embodiments, the steps of imaging the sample and imaging the population of particles are done simultaneously by fluorescence microscopy.

In some embodiments, the step of imaging the population of particles to provide a particle image is done prior to contacting the surface of the sample with a population of particles. For example, the population of particles may be fixed or trapped in a particle-receiving surface of a particle holder substrate, and the particles on the particle holder substrate are imaged to provide a particle image.

The phrase “wherein the distinguishing trait and spatial positioning of the particles of the at least 3 distinguishable subpopulations can be determined relative the surface of the sample” is intended to mean that after the sample has been imaged and the population of particles has been imaged, it is possible to determine the position of each particle of the distinguishable subpopulations with respect to the sample, and to determine into which distinguishable subpopulation each of those particles falls (e.g., small green particle, large red particle, etc.). Thus, the imaging technique must allow the determination of the distinguishing trait of each particle of the distinguishable subpopulations and the spatial position. In some embodiments, the imaging technique may also allow the determination of the spatial position of each particle of a non-distinguishable subpopulation (i.e., the spatial position of all of the particles can be determined).

The skilled person will recognise that in embodiments where all of the particles fall into a distinguishable subpopulation, the distinguishing trait and spatial positioning of each particle can be determined relative the surface of the sample. The skilled person will further recognise that in embodiments where there is a subpopulation of non-distinguishable particles and only the distinguishable subpopulations are imaged, the distinguishing trait and spatial positioning of the particles of the distinguishable subpopulations can be determined relative the surface of the sample, however the spatial positions of the particles of the non-distinguishable subpopulation is not determined relative to the surface of the sample. The skilled person will further recognise that in embodiments where there is a subpopulation of non-distinguishable particles and both the distinguishable and non-distinguishable subpopulations are imaged, the distinguishing trait and spatial positioning of the particles of the distinguishable subpopulations can be determined relative the surface of the sample, and the spatial positions of the particles of the non-distinguishable subpopulation can be determined relative to the surface of the sample.

In some embodiments, the sample image is taken before the surface of the sample is contacted by the population of particles, and the particle image is taken after the surface of the sample is contacted by the population of particles. In these embodiments, the sample image may be taken in the same field as the particle image. Overlaying the sample image with the particle image allows the determination of the spatial positioning of each particle (with its distinguishing trait) with respect to the surface of the sample. For example, if the sample is a 10×10 grid with corresponding grid coordinates (x, y), it will be possible to determine if there is small green particle at coordinate (5, 6) by viewing the overlain sample and particle images. In other embodiments, guides can be used to ensure that the particle image is overlaid correctly over the sample image. For example, the substrate and/or the particle holder substrate may comprise alignment markers that, when imaged, facilitate the overlaying of the particle image and the sample image. In some embodiments, the alignment markers are fiducials etched or tagged on the surface of the substrate and/or the particle holder substrate. In some embodiments, correct alignment is sensed by an optical or electrical sensor.

In some embodiments, the surface of the sample is contacted by the population of particles and subsequently the sample image and particle image are taken. In these embodiments, the sample image may be taken in the same field as the particle image to that the sample image can simply by overlaid with the particle image in order to determination of the spatial positioning of each particle (with its distinguishing trait) with respect to the surface of the sample. In other embodiments, guides can be used to ensure that the particle image is overlaid correctly over the sample image.

In various embodiments, the sample image and the particle image are taken before the surface of the sample is contacted by the population of particles. In these embodiments, guides can be used to ensure that the particle image is overlaid correctly over the sample image.

### Step (e)

Step (e) of the method involves capturing target biomolecules from the sample to the binding molecules, such that target biomolecules that are in close proximity to a particle bind to that particle. The term “capturing” is intended to mean that target biomolecules from the sample bind to the binding molecules through a suitable binding mechanism.

A key concept of the invention is that cells of the sample will contain and/or release target biomolecules; these biomolecules will diffuse a short distance from the cell before encountering a particle with binding molecules, and will bind to said binding molecules, therefore becoming captured by that particle. When the number/density of particles increases, there is a greater likelihood that a biomolecule will be captured by a particle in its proximity. When the resolution of the method reaches single cell resolution (e.g., there is approximately 1 particle per cell in the sample), there is confidence that a captured biomolecule on a particle originated from a cell that is positioned close to that particle. The term “close proximity” is therefore intended to mean target biomolecules within the local neighbourhood of a particle. Other terms that may be used are “in proximity”, “proximate to”, “close to”, “near to” and “within diffusion distance of”. In some embodiments, close proximity is within 20 μm of a particle. In some embodiments, close proximity is within 15 μm of a particle. In some embodiments, close proximity is within 10 μm of a particle. In some embodiments, close proximity is within 5 μm of a particle.

In some embodiments, the method further comprises the step of mobilising the target biomolecules by permeabilization. For example, the cells within the cell or tissue sample may be permeabilized (using, e.g., pepsin, physical slicing, or thermal rupture) to release the target biomolecules from inside the cells such that the target biomolecules can bind to their local particle(s). In various embodiments, the method further comprises the step of applying a binding buffer to the sample to encourage target biomolecule-particle interactions.

### Step (f)

Step (f) of the method involves removing the population of particles from the surface of the sample. The term “removing” is understood to mean collecting and retaining the population of particles from the surface of the sample such that substantially all of the particles have been separated from the surface of the sample. Preferably, at least 70% of the particles are removed from the surface of the sample. More preferably, at least 80% of the particles are removed from the surface of the sample. Even more preferably, at least 90% of the particles are removed from the surface of the sample. Even more preferably, at least 95% of the particles are removed from the surface of the sample. Even more preferably, at least 99% of the particles are removed from the surface of the sample. Most preferably, all (100%) of the particles are removed from the surface of the sample.

Removing the particles can be achieved using any suitable method that allows for collection and retention of the particles. For example, the population of particles may be removed by washing (e.g., in a wash buffer) to collect the population of particles (e.g., in the wash buffer). A washing step may comprise soaking the substrate in a wash buffer and collecting the particles from the wash buffer, and/or circulating a wash buffer over the substrate and collecting the particles from the wash buffer. In some embodiments, the population of particles is removed using a vacuum. In various embodiments, the population of particles is removed by directing pressurised fluid (e.g., wash buffer or a gas such as air) onto the population of particles. In certain embodiments, the population of particles is magnetic and is removed by magnetism (e.g., using a magnet to collect the particles, or removing a magnetic field to detach the particles from a particle holder substrate). In particular embodiments, the population of particles and the sample is removed simultaneously, and the population of particles is subsequently separated from the sample.

In some embodiments, the method further comprises a step of sorting the population of particles into the at least 3 distinguishable subpopulations based on the distinguishable trait. In embodiments where there is a non-distinguishable subpopulation, the method may comprise a step of sorting the population of particles into the at least 3 distinguishable subpopulations and the non-distinguishable subpopulation based on the distinguishable trait. In some embodiments, the sorting is done by a cell sorting method, such as fluorescence activated cell sorting (FACS) or magnetic activated cell sorting (MACS). Sorting into the subpopulations provides the advantage that the profiling step can be done on individual subpopulations, rather than the bulk population, which reduces the computational burden of the assigning step. In these embodiments, typically the particles each comprise a unique particle identifier tag. The skilled person will recognise that in embodiments where a higher number of distinguishable subpopulations is used, the sorting step may comprise sorting the population of particles into those distinguishable subpopulations (for example, in embodiments where at least 5 distinguishable subpopulations are used, the method may further comprise a step of sorting the population of particles into the at least 5 distinguishable subpopulations based on the distinguishable trait).

In some embodiments, the method further comprises a step of sorting the population of particles into individual particles. In some embodiments, the method further comprises a step of sorting the population of particles into droplets for single cell library preparation or ddPCR. In some embodiments, the sorting is done by a cell sorting method, such as fluorescence activated cell sorting (FACS). Sorting into individual particles or droplets provides the advantage that the profiling step can be done on individual particles as a single cell event, rather than the bulk population or subpopulations, which reduces the computational burden of the assigning step.

In other embodiments, individual particles are removed one at a time and profiled separately. This provides the advantage that the profiling step can be done on individual particles as a single cell event, rather than the bulk population or subpopulations, which reduces the computational burden of the assigning step.

### Step (g)

The profiling data may be any suitable data relating to the cell or tissue sample for mapping to the spatial information. For example, the profiling data may be sequencing data relating to the biological material within the cell or tissue sample. In some embodiments, the profiling data is sequencing data of transcriptomes, proteomes or epigenomes of the biological material.

The profiling step may be any suitable method that generates profiling data corresponding to each particle. In some embodiments, the profiling step is a step of generating sequence data corresponding to each particle (e.g., transcriptome, proteome or epigenome sequence data). In some embodiments, the profiling step comprises determining the sequence of the bound target biomolecules using RNA sequencing, qPCR or mass spectrometry. In various embodiments, the profiling step comprises determining the sequence of the bound target biomolecules using RNA sequencing. In many embodiments, determining the sequence of the bound target biomolecules is achieved using next generation sequencing, long read sequencing, epigenetic sequencing (e.g., bisulfite sequencing), qPCR or mass spectrometry.

In embodiments where the particles comprise a unique particle identifier tag and/or a trait identifier tag, the profiling step may further comprise profiling the unique particle identifier tags and/or trait identifier tags bound to the particles. In some embodiments, the profiling step comprises determining the sequence of the unique particle identifier tags and/or trait identifier tags bound to the particles using sequencing, qPCR or mass spectrometry. In various embodiments, the profiling step comprises determining the sequence of the unique identifier tags and/or trait identifier tags bound to the particles using sequencing. In many embodiments, determining the sequence of the unique identifier tags and/or trait identifier tags bound to the particles is achieved using next generation sequencing, long read sequencing, or epigenetic sequencing.

In some embodiments, the method further comprises one or more steps of preparing the target biomolecules for profiling. For example, the method may comprise a step of releasing the target biomolecules from the particles and collecting the target biomolecules. The method may comprise a step of extracting nucleic acids (e.g., reverse transcribing RNA to DNA, and/or generating dsDNA from ssDNA and/or bisulfite treatment). The method may comprise a step of library quality control. The method may comprise a step of library preparation (e.g., fragmentation of target biomolecules into varying sizes, end repair or A-tailing and ligation of platform-specific adapters to the library). The method may comprise a step of library amplification and/or enrichment (e.g., hybrid capture enrichment or amplicon-based enrichment). The method may comprise a step of library quantification.

Typically, the method does not comprise in-situ sequencing.

### Step (h)

A key concept of the invention is that target biomolecules will bind to particles in their local neighbourhood. Two particles in the same neighbourhood (i.e., in close proximity) will have similar bound target biomolecules. Two particles in completely separate neighbourhoods are very unlikely to have similar bound target biomolecules. When the bound target biomolecules of particles are profiled, a profile (or a partial profile) of a particle can be compared with the other particle profiles (or partial profiles), and particles with highly similar profiles (or partial profiles) are highly likely to be neighbours (i.e., can be presumed to be neighbours). Typically, a “partial profile” of a particle comprises profiling data corresponding to identifier tags, if present (e.g., unique spatial identifier tag, trait identifier tag, and/or unique particle identifier tag), and profiling data corresponding to a subset of the bound target biomolecules. This is otherwise known as a “particle signature”.

Any suitable method of determining the similarity of pairs or pluralities of particles may be used. For example, clustering analysis, Pearson correlation coefficient analysis or Euclidean distance analysis may be used.

The term “pairs of particles” is not meant to imply that the particles within the pair are matched or twinned in any way. This term simply means a group of two particles. Advantageously, a particle can be compared against the other particles in turn to assess how similar their profile data are. The term “pluralities of particles” is not meant to imply that the particles within the plurality are matched in any way. This term simply means a group of two or more particles. Advantageously, a particle can be compared against multiple other particles simultaneously to assess how similar their profile data are.

In some embodiments, the step of calculating a similarity score for each pair of particles involves assessing the similarity of the profile data of a first particle with the profile data of a second particle and assigning a similarity score based on how similar the profile data of the first particle is to the second particle, and repeating for each pair of particles.

In some embodiments, the step of calculating a similarity score for each pair of particles involves calculating the Eucledian distance, the Manhattan distance, the mahalanobis distance, the pearson correlation, the uncentered correlation, the Spellman rank correlation or the absolute or square correlation. These techniques are known in the art and are discussed in D'haeseleer, P. (“How does gene expression clustering work? Nature Biotechnology 2005 23(12) 1499-1501), which is hereby incorporated by reference in its entirety.

In some embodiments, a similarity score is calculated for 100% of the pairs of particles. In various embodiments, a similarity score is calculated for 99% of the pairs of particles. In some embodiments, a similarity score is calculated for 98% of the pairs of particles. In some embodiments, a similarity score is calculated for 95% of the pairs of particles. In some embodiments, a similarity score is calculated for 90% of the pairs of particles. In some embodiments, a similarity score is calculated for 80% of the pairs of particles. In some embodiments, a similarity score is calculated for 70% of the pairs of particles.

In some embodiments, the method further comprises the step of applying a similarity score threshold, such that a pair of particles having a similarity score below the threshold are not considered spatially located within a same neighbourhood, and a pair of particles having a similarity score above the threshold are considered spatially located within a same neighbourhood.

In some embodiments, the similarity score is a score out of 100. In various embodiments, the similarity score threshold is at least 80. In various embodiments, the similarity score threshold is at least 85. In various embodiments, the similarity score threshold is at least 90. In various embodiments, the similarity score threshold is at least 95. In various embodiments, the similarity score threshold is at least 98.

### Step (i)

As discussed above, particles with highly similar profiles (i.e., having a high similarity score) are highly likely to be neighbours (i.e., can be presumed to be neighbours). Accordingly, groups of similar profiling data can be presumed to originate from a local neighbourhood of particles, and can be assigned to a spatial neighbourhood of particles in the particle image. For example, 5 profiles were assessed to be highly similar and therefore in the same neighbourhood; these 5 profiles corresponded to a blue bead, a green bead, a red bead, a yellow bead and a purple bead (e.g., they comprised a barcode corresponding to colour, or the particles were sorted into colour by FACS), and they could be assigned to a group of 5 particles (blue, green, red, yellow and purple) in the particle image which were in the same neighbourhood.

The term “assigning the profiling data” is intended to mean that the profile data from a particle is mapped onto a specific particle in the particle image or an inferred particle position, such that it can be interpreted that that profile data originated from that specific particle or a particle inferred to be at that location, and ergo from that specific spatial position, which should correspond with a particular cell in the sample image.

Instead of the term “spatial position”, the term “grid coordinate” could also be used.

In some embodiments, the method may comprise one or more additional features that decrease the computational burden of the assigning step. These are discussed in further detail below.

### Unique Spatial Identifier Tags

In some embodiments, the particle-receiving surface of the particle holder substrate comprises a plurality of distinct spatial areas, each distinct spatial area comprising a unique spatial identifier tag. By unique spatial identifier tag it is meant a tag or barcode that allows each distinct spatial area to be uniquely identified within the plurality of spatial areas. For example, the particle-receiving surface may be divided into spatial areas (i.e., zones), with each area having a distinct tag which allows the different areas to be distinguished. The unique spatial identifier tags may be deposited onto the particle-receiving surface of the particle holder substrate using any suitable method that deposits the unique spatial identifier tags into the plurality of distinct spatial areas. For example, the unique spatial identifier tags may be printed onto the particle-receiving surface of the particle holder substrate. As a further example, the unique spatial identifier tags may be flowed over the surface of the particle holder substrate, spin coated or applied via electrophoresis or diffusion. The unique spatial identifier tags provide the advantage that the particles positioned in a certain spatial area will bind to the unique spatial identifier tag associated with that area. When the particle is profiled, the profiling data will include the profile of the unique identifier tag. It is then possible to map the profiling data of that particle back to the spatial area of the particle holder substrate associated with that unique identifier tag. When determining similarity of pairs of particles, it is only necessary to compare pairs of particles within the same spatial area, reducing the computational burden. Similarly, when assigning the profiling data back to a spatial position, this step is less computationally intense because fewer spatial positions are being selected from (i.e., only the spatial positions within the spatial area associated with the unique identifier tag).

In some embodiments, each unique spatial identifier tag is a degenerate or semi-degenerate nucleotide sequence 5 to 10 bp in length. Each unique spatial identifier tag may comprise DNA, RNA, synthetic oligonucleotides or a combination thereof. Preferably, each unique spatial identifier tag comprises DNA. More preferably, each unique spatial identifier tag consists of DNA. Each unique spatial identifier tag may be single stranded or double stranded. The unique spatial identifier tag may be incorporated into a sequencing adapter. The sequencing adapter may be 300-15,000 bp in length, preferably 300-500 bp in length.

In some embodiments, the plurality of distinct spatial areas comprises at least 1 distinct spatial area per square millimetre of sample. In some embodiments, the plurality of distinct spatial areas comprises at least 10 distinct spatial areas per square millimetre of sample. In some embodiments, the plurality of distinct spatial areas comprises at least 100 distinct spatial areas per square millimetre of sample. In some embodiments, the plurality of distinct spatial areas comprises at least 1000 distinct spatial areas per square millimetre of sample. In some embodiments, the plurality of distinct spatial areas comprises 1-1000 distinct spatial areas per square millimetre of sample.

### Trait Identifier Tags

In various embodiments, each particle comprises a trait identifier tag which corresponds to the distinguishable trait of that particle. By trait identifier tag, it is meant a tag or barcode that allows the distinguishable trait of a distinguishable subpopulation of particles to be uniquely identified within the population of particles. For example, a distinguishable trait of a distinguishable subpopulation of particles may be the fluorescent tag colour green and the particle size small; each particle within this distinguishable subpopulation can be tagged with a trait identifier corresponding to “green small”.

In some embodiments, each trait identifier tag is a degenerate or semi-degenerate nucleotide sequence 3 to 10 bp in length. In some embodiments, each trait identifier tag is a degenerate or semi-degenerate nucleotide sequence 4 to 8 bp in length. Each trait identifier tag may comprise DNA, RNA, synthetic oligonucleotides or a combination thereof. Preferably, each trait identifier tag comprises DNA. More preferably, each trait identifier tag consists of DNA. Each trait identifier tag may be single stranded or double stranded.

Using a trait identifier provides the advantage that the assigning step is computationally less intense because the trait identifier (as part of the profiling data of a particle) restricts the possible spatial positions that the data can assigned to. For instance, profiling data with the trait identifier corresponding to a small green particle can only be assigned back to the small green particles in the particle image.

In some embodiments, the particles comprise releasable trait identifier tags and trait identifier tag binding molecules that bind to released trait identifier tags. In these embodiments, the method further comprises the steps of: releasing the releasable trait identifier tags from the particles; and capturing the released trait identifier tags to the trait identifier tag binding molecules, such that trait identifier tags that have been released in close proximity to a particle bind to that particle; wherein the assigning step is further based on the captured trait identifier tag profile of each particle. This modification provides the advantage that tags corresponding to the trait of the particle can be triggered to be released from the particle, which then bind to other particles in the local neighbourhood; after this step, a particle will be bound to target biomolecules released from local cells, and tags released from local particles indicating their trait. When a particle is then profiled, it is easier to assign the particle profile to a particular spatial location as one is (i) comparing the similarities of particle profiles to identify neighbours, and (ii) assessing the particle acquired tag profile to identify the trait of neighbours.

### Unique Particle Identifier Tags

In some embodiments, each particle comprises a unique particle identifier tag. By unique particle identifier tag it is meant a tag or barcode that allows each particle to be uniquely identified within the population of particles. This provides the advantage that the particles may be profiled in bulk and the profiling data corresponding to each particle can be uniquely identified.

In some embodiments, each unique particle identifier tag is a degenerate or semi-degenerate nucleotide sequence 8 to 15 bp in length. Each unique particle identifier tag may comprise DNA, RNA, synthetic oligonucleotides or a combination thereof. Preferably, each unique particle identifier tag comprises DNA. More preferably, each unique particle identifier tag consists of DNA. Each unique particle identifier tag may be single stranded or double stranded.

In preferable embodiments, the particles do not comprise a unique particle identifier tag. In these embodiments, each particle cannot be individually distinguished. This reduces the burden on the number of unique barcodes necessary for the method to work.

Where landmark particles are used, in some embodiments the landmark particles each comprise a unique particle identifier tag and the non-landmark particles do not comprise a unique particle identifier tag.

### 3D Assignment

In some embodiments, the sample is a slice of a tissue, wherein steps (a), (b) and (d)-(g) of the method are repeated on a further slice of the tissue to generate profiling data corresponding to the further slice. In these embodiments, the calculating step is further based on the profiling data corresponding to the further slice. This modification to the method provides the advantage that particle profiles from one slice can be compared to particle profiles from the other tissue slices; highly similar profiles across slices will identify a similar or identical spatial position of the particles; the sequence of traits of the particles across the layers can therefore be used to assign those particles to a spatial position in the particle image. It can be inferred that the profiles of those particles corresponding to the same spatial position also correspond to the cell present in that spatial position on the same image.

In various embodiments, the sample is a plurality of slices of tissue less than 10 μm thick.

In various embodiments, the sample is a plurality of slices of tissue less than 9 μm thick.

In various embodiments, the sample is a plurality of slices of tissue less than 8 μm thick.

In various embodiments, the sample is a plurality of slices of tissue less than 7 μm thick.

### Sequential Removal

In some embodiments, the step of removing the population of particles from the surface of the sample involves removing all of the particles in a single step. In alternative embodiments, the step of removing the population of particles from the surface of the sample involves removing particles in sequential steps. For example, the particles may be removed from distinct areas of the surface of the sample in 1-20 steps. In some embodiments, the particles are removed from distinct areas of the surface of the sample in 2-15 steps. In various embodiments, the particles are removed from distinct areas of the surface of the sample in 5-10 steps. In some embodiments, the particles are sequentially removed using vacuum-induced sequential release. In various embodiments, distinct spatial areas of particles are released using light activation or magnetism.

Sequential removal of the particles provides the advantage that the sequentially removed groups of particles can be profiled separately. Accordingly, when assigning profiling data of a particle to a spatial position, the computational burden is reduced because the possible spatial positions are restricted to the spatial area from which that group of particles was removed. For example, if a first group of particles was removed from a top left quadrant and subsequently profiled, the profiling data corresponding to a particle in this group can only be assigned back to a spatial position within the top left quadrant.

### Landmark Particles

In some embodiments, the population of particles comprises landmark particles and non-landmark particles, wherein the landmark particles comprise the at least 3 distinguishable subpopulations (in preferable embodiments, at least 5 distinguishable subpopulations), and wherein the non-landmark particles do not have a distinguishable trait that can be determined by imaging. The term “landmark particle” is intended to mean a particle having a trait that is distinguishable by imaging and distinguishes the landmark particle from a non-landmark particle (i.e., a non-distinguishable particle) and from landmark particles from another distinguishable subpopulation (e.g., a blue landmark bead is distinguishable from a green landmark bead and a non-landmark bead). Landmark particles may also be referred to as “distinguishable” or “trait” particles.

In these embodiments, each landmark particle (and associated profiling data) is assigned to a spatial position of a particle in the particle image. In some embodiments, the landmark particle assigning step is based on the particle image alone (e.g., if each particle has a different distinguishable trait which is discernible in the particle image, each particle can be precisely mapped to a particle in the particle image due to the presence of that trait). In some embodiments, the landmark particle assigning step is based on the particle image and the similarity scores of the profiling data. For example, the profiling data of all of the landmark particles may be compared as described in step (h) to calculate similarity scores for the landmark particles, and then each landmark particle is assigned to a spatial position based on neighbourhood presumption and the distinguishable trait. This results in all of the landmark particles being assigned to spatial positions in the particle image as a first step.

The profiling data of a non-landmark particle may then be compared to each landmark particle and a similarity score calculated. Particles with highly similar profiles (i.e., having a high similarity score) are highly likely to be neighbours. Particles with decreasingly similar profiles are more likely to be further away in physical space. This profile distance and physical distance correlation may thus be used to assign a non-landmark particle to a spatial location based on the similarity scores of the non-landmark particle with the landmark particles.

The profiling data of each remaining unassigned non-landmark particle may then be compared to each assigned landmark particle and each assigned non-landmark particle and a similarity score calculated. The profile distance and physical distance correlation may be used to assign a non-landmark particle to a spatial location based on the similarity scores of the non-landmark particle with the assigned landmark particles and assigned non-landmark particles (which act as pseudo-landmark particles once assigned).

In various embodiments, steps (h) and (i) of the method may therefore comprise:
(1) assigning the profiling data corresponding to a landmark particle to a spatial position of a landmark particle in the particle image, based on similarity scores and/or the particle image;
(2) calculating similarity scores for pairs or pluralities of landmark beads with non-landmark particles based on the profiling data;
(3) assigning the profiling data corresponding to a non-landmark particle to a spatial position of a non-landmark particle in the particle image, based on the similarity scores and/or the particle image;
(4) repeating step (3) for other non-landmark particles that have not yet been assigned a spatial position.

In various embodiments, steps (h) and (i) of the method may comprise:
(1) assigning the profiling data corresponding to a landmark particle to a spatial position of a landmark particle in the particle image, based on similarity scores and/or the particle image;
(2) repeating step (1) for unassigned landmark particles;
(3) calculating similarity scores for a first non-landmark particle with each assigned landmark particle based on the profiling data;
(3) assigning the profiling data corresponding to the first non-landmark particle to a spatial position of a non-landmark particle in the particle image, based on the similarity scores and/or the particle image;
(4) calculating similarity scores for a second non-landmark particle with each assigned landmark particle and assigned non-landmark particle based on the profiling data
(5) assigning the profiling data corresponding to the second non-landmark particle to a spatial position of a non-landmark particle in the particle image, based on the similarity scores and/or the particle image;
(6) repeating steps (4) and (5) for other non-landmark particles that have not yet been assigned a spatial position.

In some embodiments, the profiling data of each non-landmark particle is compared to the profiling data of at least three landmark particles in a triangulation method to assign the non-landmark particle to a spatial position. Advantageously, this increases the likelihood of correctly assigning the particles to a spatial position.

In some embodiments, the particle image only provides the spatial positions of the landmark particles, and does not provide the spatial positions of the non-landmark particles. In this case, the spatial positions of the non-landmark particles can be assumed to be in a grid or array formation, provided that the particles are densely packed (e.g., one particle per cell). In these embodiments, the profiling data corresponding to a non-landmark particle is assigned to an inferred spatial position of a non-landmark particle in the particle image. This provides the advantage that only the landmark particles need to be imaged, and not the entire population of particles.

The landmark particle method provides the advantage that not all of the beads need to have a distinguishable trait, such as a fluorescent tag. This makes the method cheaper and easier to manufacture. Because of the lower number of distinguishable trait particles, these can be assigned to a spatial position with a high degree of certainty, either because each landmark particle is distinguishable from each other landmark particle (and therefore only has one possible spatial position in the particle image), or because the landmark particles will be further apart from each other and therefore there will be fewer particles and fewer spatial positions to assign them to, making it more likely a particle is assigned to the correct spatial position. The landmark beads can then be used in the process of assigning the non-landmark beads to spatial positions, based on the profile distances (i.e., similarity of profile data) correlating to distance to the landmark beads in physical space. The inventors have particularly found that a triangulation method can be used to triangulate the spatial position of a non-landmark particle between three landmark particles, based on the similarity of profile data to those three landmark particles.

### Step (j)

Step (j) of the method involves providing a virtual map of the spatially resolved profiling data with respect to the sample image. As discussed above, during the assigning step, profile data originating from a specific particle is assigned to a spatial position of a particle within the particle image (or an inferred spatial position of a particle within the particle image). As the spatial positioning of the particles has been determined relative to the surface of the sample, the profiling data can be spatially resolved against the sample image. In some embodiments, the profiling data from a spatially resolved particle is assigned to a cell in the sample image.

In some embodiments, the method further comprises the step of providing a composite representation comprising the sample image and a representation of all or a portion of the profiling data, wherein the profiling data for a particular spatial location is aligned to that spatial location on the sample image.

A further aspect of the present invention provides a particle holder substrate comprising a population of particles, wherein the population of particles is randomly distributed on a particle-receiving surface of the particle holder substrate, wherein the particles comprise at least 3 distinguishable subpopulations (in preferable embodiments, at least 5 distinguishable subpopulations), wherein each distinguishable subpopulation has a distinguishable trait that can be determined by imaging, and wherein the particles comprise binding molecules that bind to target biomolecules. The description above in relation to the method of the present invention is equally applicable to the particle holder substrate aspect.

For example, in some embodiments, the population of particles is fixed to or trapped in a particle-receiving surface of the particle holder substrate (may also be referred to as a particle holder, or a particle substrate). In various embodiments, the population of particles may be trapped within cavities or wells in the particle-receiving surface of the particle holder substrate. For example, the cavities or wells may be formed by laser drilling. The population of particles may be fixed to the particle-receiving surface of the particle holder substrate by surface chemistry. In some embodiments, the population of particles is fixed by surface chemistry within cavities or wells in the particle-receiving surface of the particle holder substrate. The population of particles may be deposited to the particle-receiving surface in any suitable manner which deposits the particles randomly and with high efficiency, and with a spacing approximating the cell spacing of a cell or tissue sample. In certain embodiments, sensitivity can be improved by decreasing the spacing such that there are multiple particles per cell.

The population of particles is randomly distributed on the particle-receiving surface of the particle holder substrate. For example, the particles are not purposefully positioned at specific coordinates of the particle holder substrate. This provides the advantage that the particle holder substrate is cheaper and easier to manufacture compared to existing technologies where lawns of barcodes are printed onto slides in a non-random manner. In various embodiments, 70% of the population is randomly distributed on the particle holder substrate. In various embodiments, 80% of the population of particles is randomly distributed on the particle holder substrate. In various embodiments, 90% of the population of particles is randomly distributed on the particle holder substrate. In various embodiments, 95% of the population of particles is randomly distributed on the particle holder substrate. In various embodiments, 98% of the population of particles is randomly distributed on the particle holder substrate. In various embodiments, 99% of the population of particles is randomly distributed on the particle holder substrate. In various embodiments, 100% of the population of particles is randomly distributed on the particle holder substrate.

A further aspect of the present invention provides a system comprising a processor and a computer readable medium storing one or more instruction(s) arranged such that when executed the processor is caused to: calculate similarity scores for pairs of particles based on a set of profiling data; assign the profiling data corresponding to a particle to a spatial position of a particle in a particle image, based on the similarity scores and/or the particle image; and provide a virtual map of the spatially resolved profiling data with respect to a sample image. The description above in relation to the method of the present invention is equally applicable to the system aspect. In particular, the profiling data may be generated as described above.

### BRIEF DESCRIPTION OF THE DRAWINGS

The invention will now be described in detail by way of example only with reference to the figures in which:

FIG. 1 shows a flowchart of an exemplary embodiment of the method of the disclosure.

FIG. 2 is a graph showing the dependence of the minimum number of subpopulations (MNS) on the number of sites (NOS) in embodiments where all particles fall into a distinguishable subpopulation.

FIG. 3 shows an exemplary binding molecule that may be conjugated to a particle for use in the method of the disclosure.

FIG. 4 shows three example particle neighbourhoods. A dark blue particle with a unique particle identifier DB #1 is in the neighbourhood of 2 light blue particles, 2 yellow particles, 1 purple particle, 1 dark blue particle and 2 dark green particles. A dark blue particle with a unique particle identifier DB #2 is in the neighbourhood of 2 light green particles, 2 orange particles, 1 purple particle, 1 light blue particle, 1 yellow particle and 1 blue particle. A dark blue particle with a unique particle identifier DB #3 is in the neighbourhood of 4 dark blue particles, 2 orange particles and 2 purple particles.

FIG. 5 shows complementary methods that can be used with the present invention. (A) shows sequential release of particles from distinct spatial areas of the sample. (B) shows identification based on bead sequence in 3D. (C) shows artificially generated/amplified neighbourhood patterns. (D) shows unique spatial identifiers.

FIG. 6 shows a simulation of an exemplary assigning step of a method according to the disclosure.

FIG. 7 shows a technique of dimensionality reduction via manifold learning.

FIG. 8 shows a technique of dimensionality reduction via manifold learning.

FIG. 9 shows how an exemplary embodiment of the assigning step of the method of the disclosure, using seeded reconstruction.

FIG. 10 shows an exemplary embodiment of the assigning step of the method according to the disclosure.

FIG. 11 is a block diagram illustrating a computer system on which steps (h)-(j) of the method of the disclosure may be implemented.

### DETAILED DESCRIPTION OF THE INVENTION

The present invention uses particles (“collector particles”) which can be dispersed at densities matching the cell densities of a cell or tissue sample and are brought into contact with the sample (e.g., an intact tissue slice). The distribution of the particles can be detected by imaging. The particles can be grouped into a number of distinct distinguishable subpopulations based on a trait, and the number of traits is chosen such that statistically each particle with a given trait has a unique neighbourhood amongst the particles with similar traits and this can be used to reconstruct the spatial arrangement of the particles using the image of the particles.

Biomolecules from the sample will bind to the particles in their local neighbourhood. The profile of the biomolecules can subsequently be determined and assigned back to a particular particle in the particle image.

A major merit of the present innovation is that it allows for true single cell or subcellular resolution spatial biology, with high tissue coverage. This is made possible by the availability of particle alternatives, for example microbeads, whose size matches or can even be smaller than the size of cells in a tissue. Current technologies using sequencing for spatial biology are limited in resolution to around 5-10 cell resolution by the size of spots they can print, which greatly reduces the usability of the resulting data. The coverage of such data is also limited by the requirement for specific pitch distances between spots of capture molecules (spatial barcodes) without them merging during the printing process and becoming indistinguishable. Single cell resolution is especially important in in homogeneous tissues, where cell types vary within short distances, for example in the diverse population of cancer cells or in the brain. Furthermore, this innovation also has the potential to increase the efficiency of the detection. While spot printing is limited by the minimal distance between spots, particles can be deposited much closer to each other, which would allow for gathering information from a larger portion of the tissue.

Additionally, it is also to be appreciated that the current invention achieves this with a reduced number of labels and barcodes. It is sufficient for the current method to have 20-30 different labels and a unique barcode that is only 10-15 bases long, depending on the size of the tissue and its cell density. This greatly reduces the cost of consumables and simplifies both manufacture and the workflow, making it more suitable for automation. In particular, current methods relying on printing of spots of different barcodes face the challenge of printing more than 5000 different spots, that is time consuming and requires highly complex fluid handling systems. On the other hand, the current invention would simplify the preparation of the detection surface by allowing the particles to be mixed and spread out randomly in a single step over the detection surface. This reduced complexity is made possible by the computational methods that reconstruct the spatial arrangement. This method permits the shift of complexity from the hardware to the software. With the abundance of data handling platforms and the current trend towards the use of these, this increases the usability of any platform using this innovation.

The present invention provides the advantage that a unique identifier is not required on every particle. Instead, a simple image is taken of the particles in relation to the sample, and neighbourhood mapping is used to assign particles (and associated profile data) in the sequence space to a spatial position in the physical space. This method does not require in situ sequencing, and thus avoids the complexities of multiple imaging steps separated by reagent changes. In addition, the present invention can be used on a significantly larger sample compared to in situ sequencing methods which are limited by area.

As it will be demonstrated in the following examples, the assignment of the profiling data back to a particular particle with a particular spatial position can be carried out via, for example, a dimensionality reduction step followed by a sequential reconstruction algorithm. The dimensionality reduction algorithms are widely used in the machine learning and data analysis community whereas the sequential reconstruction can be tailored to the quality and structure of the tissues of interest. The sequencing can, for example, be performed on Illumina, Ultima, Pacific Biosciences instrumentation, Oxford Nanopore platforms or other commercially available sequencers.

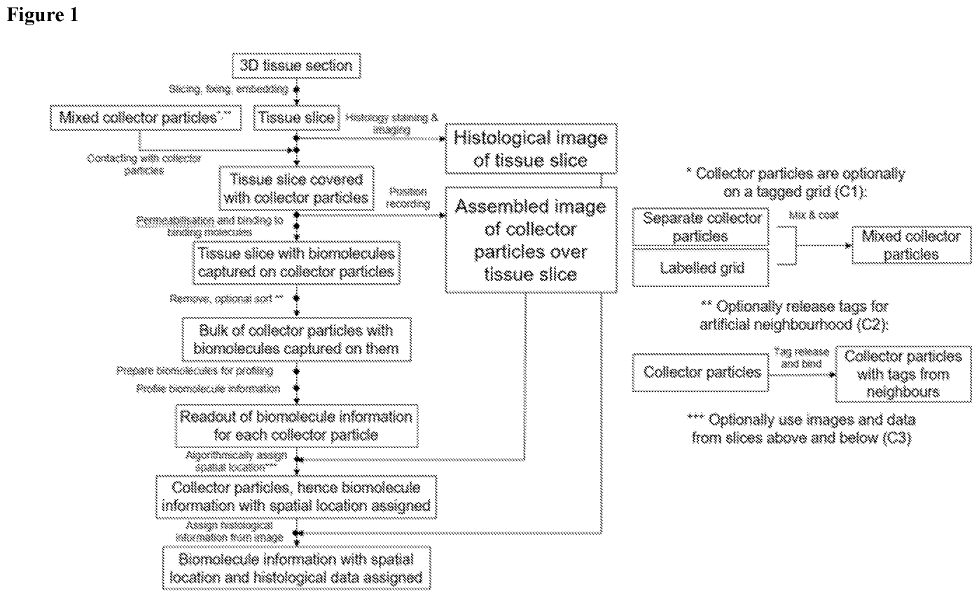

The current invention achieves spatially resolved profiling of biological information in a sequence of steps at a single cell or subcellular resolution with only a small number of unique tags needed. FIG. 1 details a number of the steps according to an exemplary embodiment of the invention:
1. Tissue sections are prepared for analysis (e.g., FFPE or fresh frozen); or prep-prepared tissue sections are obtained. This may involve slicing, fixed and embedding of the tissue section.
2. The tissue section is stained (if desired) and imaged to provide an image of the tissue section (sample image).
3. The tissue section is brought into contact with particles.
   a. The particles may be present on a tagged grid (see C1 method detailed below);
   b. The particles may be triggered to release trait identifier tags, which bind to neighbouring particles (see C2 method detailed below);
4. Biomolecules from the tissue section bind to particles in close proximity via binding molecules on the particle.
5. The position of each particle relative to the sample is determined by imaging, thereby providing an assembled image of the particles over the tissue section.
6. The particles are removed from the surface and collected in bulk.
   a. Optionally, the particles are sorted into groups based on their trait.
7. The bound biomolecule content of each particle is profiled via biomolecule analytical techniques (e.g., sequencing).
8. The profile of each particle is algorithmically assigned back to a spatial position on the assembled image via local neighbourhood mapping.
   a. The method is optionally performed on multiple tissue slices of the same tissue sample (see C3 method below) to reduce the computational burden of the algorithmic step

The key element of the invention is that it is possible to infer spatial neighbourhood information from the biomolecule profile (i.e., signature) collected from each particle and the introduced labels recorded at the imaging step to create unique neighbourhood patterns that can be used to locate the position of the particles.

During the development of the present invention, the inventors discovered that only a small number of distinguishable subpopulations are needed for the method to work. When all of the particles fall into a distinguishable subpopulation, this number, denoted minimal number of subpopulations (MNS) is given by equation:

$$\frac{S}{T} \leq \frac{( T + Z - 1 ) !}{( T - 1 ) ! Z !}$$

where S is the total number of sites, T is the number of distinguishable subpopulations and Z is the number of neighbours in a neighbourhood.

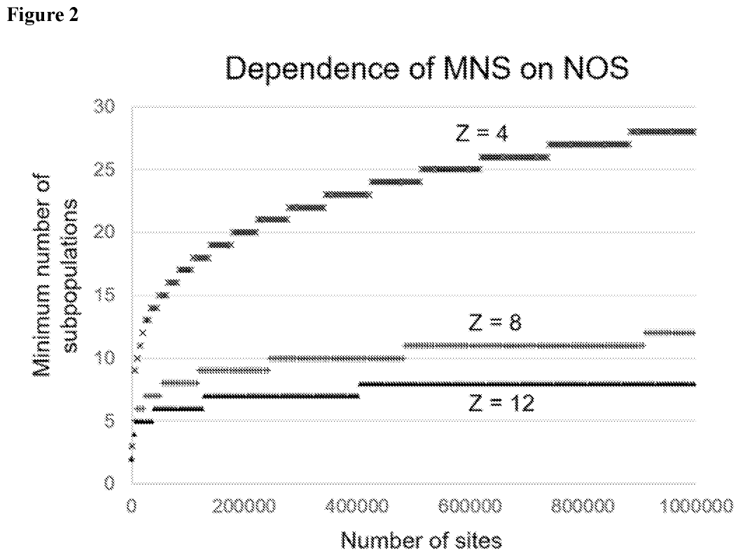

Accordingly, as shown in FIG. 2, the MNS depends on the number of sites (NOS) in consideration and the size of the neighbourhood that can be used for analysis. For a typical sample containing around one million cells (sites) and immediate neighbourhood of 4 cells, the MNS is 28; for an immediate neighbourhood of 8 cells, the MNS is 12; for an immediate neighbourhood of 12 cells, the MNS is 8.

This finding is advantageous as it limits the number of different particle traits to not more than 30 (in contrast to the 5000+ used in today's printing based spatial sequencing technologies). Needing only a small number of distinct traits means reduced reagent costs, simpler manufacturing processes and possibly improved usability while improving the current limits of achievable spatial resolution. Furthermore, this invention can also be used to collect multiple types of biological materials (e.g., RNA, DNA, proteins, lipids, metabolites) and investigate different properties of these (e.g., DNA methylation marks, post translation modifications, RNA epigenetic marks).

The current invention could be used in true discovery applications, diagnostic and fundamental research settings. The invention could be applied to de novo sequencing approaches where no prior knowledge of the biomolecules of interest is required.

In diagnostic environments, a targeted plex range of around 1-10,000 could be useful. Within the framework of the current invention this can be achieved by attaching target biomolecule binding molecules (e.g., specific RNA/DNA segment complements, antibodies or proteins) to the particles. In diagnostic settings, this innovation can thus be used, for example, to rapidly determine the spatial expression patterns of a known and validated biomarkers in cancer biopsy, assisting the choice of chemotherapy drug or in agricultural setting to discover underlying mechanistic causes of disease.

Ultimately, this invention could not only find use in the aforementioned cases—disease screening in diagnostic and agricultural settings and applied research for drug development but also in more fundamental research areas. One example field is developmental biology where understanding the spatial gene activation patterns is one of the main aims.

### Target Biomolecule Binding

One of the key elements of this invention is that biomolecules from the sample are collected by the population of particles that is in contact with the sample. This contrasts to prior techniques widely used in bulk phase applications where biomolecules are collected onto a slide with lawns of printed barcodes.

Profiling of target biomolecules from the sample is possible because the particles comprise binding molecules for the target biomolecules. If desired, unwanted target biomolecules may be removed or deactivated prior to contact with the particles. All mRNA transcripts can be collected, for example, via the hybridisation of the polyA region present in all mRNA molecules to a polyT sequence present on the particle. Global DNA profiling could be performed, for example, via the hybridisation of DNA molecules to a polyN sequence on the particles, with unwanted RNA and proteins being removed via the application of RNAses and Proteases, respectively.

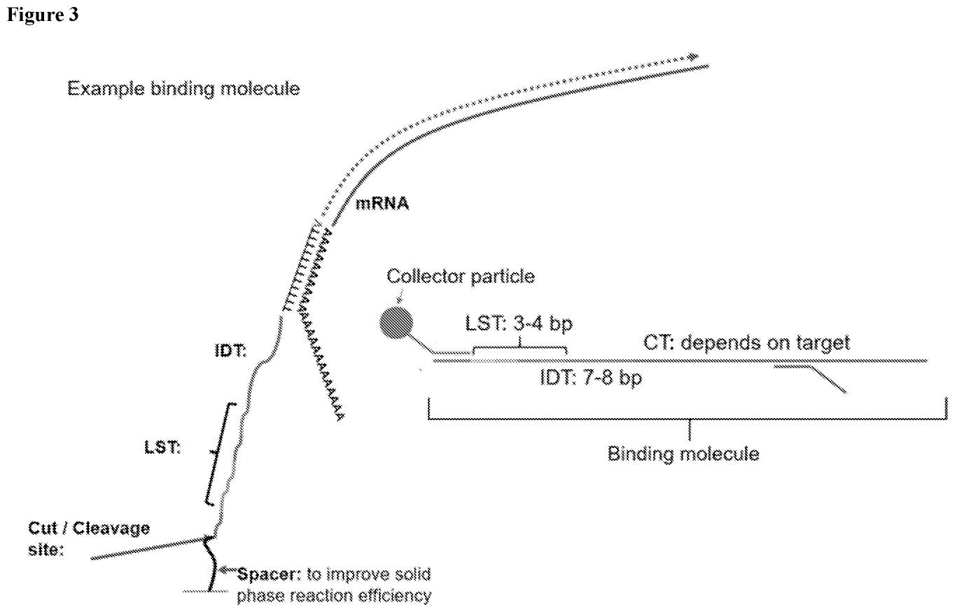

FIG. 3 shows an exemplary binding molecule present on the surface of a particle. The binding molecule has three different regions: a particle trait identifier (also referred to as a label specific tag (LST)), a unique particle identifier (also referred to as an identifier tag (IDT)), and a collector tag (CT). In alternative embodiments, the particle may not have a particle trait identifier or a unique particle identifier.

The particle trait identifier indicates the trait (or at least one characteristic) of the particle. For example, when coloured particles are used, the particle trait identifier indicates (after readout) the colour of the particle: a green particle will have a particle trait identifier that indicates the particle is green. The particle trait identifier is typically a DNA barcode 3-4 bp in length.

The unique particle identifier provides a unique barcode for the particle among the particles within the same distinguishable subpopulation (e.g., green particle #2, green particle #5). The unique identifier is typically a DNA barcode 7-8 bp in length.

The collector tag is a binding partner for the target biomolecules. The nature of the collector tag depends on the target biomolecules. For example, when the target biomolecules are mRNA, the collector tag may be a polyT tail which non-specifically binds to the polyA tail of mRNA. As a further example, when the target biomolecules are proteins, the collector tag may be a target specific DNA segment followed by a capture antibody.

When the particles are removed from the tissue (e.g., by washing), the biomolecule profile of each particle can be determined in an analytical step. It is preferred to use short read sequencing to determine the biomolecule profile. Alternative methods to sequencing include qPCR, mass spectrometry or long read sequencing techniques.

### Neighbourhood Information

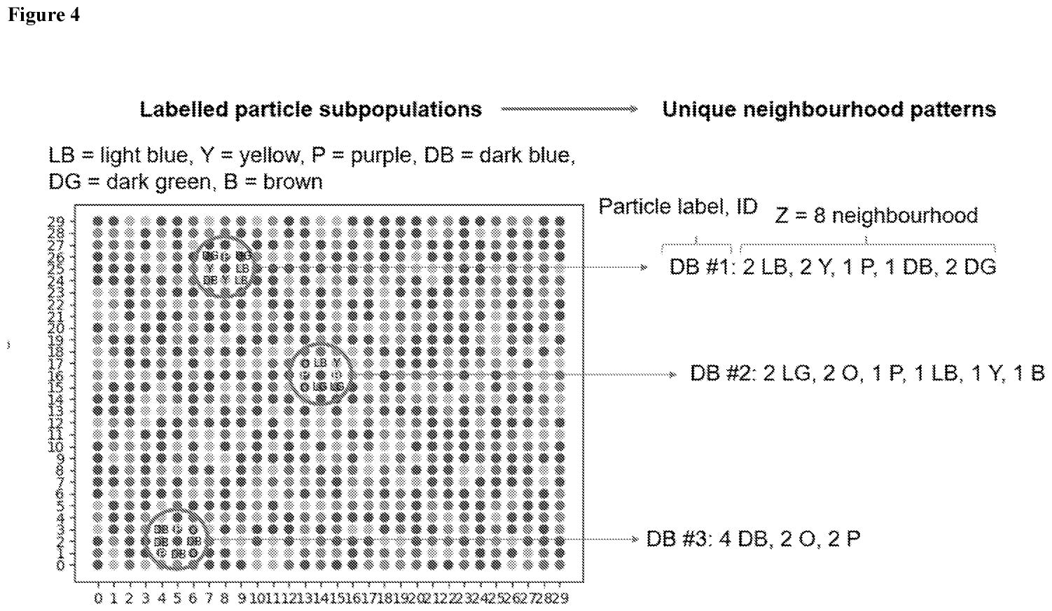

Another key element of this invention is that using a small number of different particle populations (traits and optionally identifier tags) complemented by the neighbourhood information deducible from the biomolecule profile (e.g., gene expression signature), it is possible to allocate back the information from the biomolecule profiling to the spatial position. This is possible because multiple distinguishable subpopulations of particles with different traits creates unique neighbourhood patterns. This is shown in FIG. 4, where each particle comprises a unique particle identifier and a trait identifier and can therefore be sequenced in bulk. For each particle, it is possible to identify the colour of the particle (LB, Y, P, DB, DG or B) based on the sequence information of the trait identifier. It is also possible to identify the unique particle identifier of each particle (e.g., DB #1). When the similarity of particle profiles is assessed, it is possible to deduce that DB #1 is in the same neighbourhood as 2 LB, 2 Y, 1 P, 1 DB and 2DG. Based on this information, it is possible to assign the profile information of DB #1 to the spatial position corresponding to coordinate (8, 25) on the assembled sample-particle image. It can therefore be inferred that the cell present at coordinate (8, 25) has the profiling data of DB #1. This can be repeated for the data for each particle, such that each particle profiling data is assigned a spatial position on the assembled sample-particle image.

Accordingly, this invention uses the correlation between the spatial distance of particles and the ‘profile distance’ or particles in order to assign profile information of a particle back to a spatial position. In different terms, the closer in similarity the profile of two particles, the greater the likelihood that these two particles are spatially close.

### Example 1

The following exemplary protocol is provided. In this example, the particles do not have unique identifier tags. In this example, all of the particles fall into a distinguishable subpopulation.

### Preparation of Tissue

Prepare tissue for subsequent analysis using routine methods (e.g., FFPE or fresh frozen. Stain tissue using routine histological methods.

### Sample Imaging

Image the tissue using an appropriate technique (e.g., bright field microscopy).

### Sample-Particle Contact

Identify the number of particles (e.g., microbeads) required for the tissue section (factoring in section area and cell density) and the number of particle subpopulations required (e.g., 10⁶ particles, 5 different sizes of particle (a, b, c, d, e), 5 different fluorescent colours). Apply the population of particles to the tissue section. Biomolecules from the tissue section will bind to their closest particle via binding molecules on the particle.

### Particle Imaging

Image the particles on the sample using an appropriate technique (e.g., fluorescence microscopy). This image provides a spatial map of all of the particles relative to the sample, and the particle trait of each particle (e.g., green size a).

### Particle Collection

The particles are removed from the surface and collected in bulk. The particles are subsequently sorted into individual particles.

### Particle Profiling

Each particle is put into a single cell library preparation protocol where each particle is treated as a unique reaction in a droplet, well or similar. During the single cell library preparation process, a unique particle identifier barcode is added to the biomolecule content of each particle. The bound biomolecule content of each particle is profiled via biomolecule analytical techniques (e.g., sequencing).

### Assigning Particle Spatial Positions

Similarity between biomolecule profiles of particles is used to assign a spatial position to each particle, based on the particle image via local neighbourhood mapping. The unique particle identifier barcode enables the associated sequencing reads to be reassigned to the correct particle bioinformatically.

### Example 2

The following exemplary protocol is provided. In this example, the particles have unique identifier tags.

### Preparation of Tissue

Prepare tissue for subsequent analysis using routine methods (e.g., FFPE or fresh frozen. Stain tissue using routine histological methods.

### Sample Imaging

Image the tissue using an appropriate technique (e.g., bright field microscopy).

### Sample-Particle Contact

Identify the number of particles (e.g., microbeads) required for the tissue section (factoring in section area and cell density) and the number of particle subpopulations required (e.g., 10⁶ particles, 5 different sizes of particle (a, b, c, d, e), 5 different fluorescent colours). Apply the population of particles to the tissue section. Biomolecules from the tissue section will bind to their closest particle via binding molecules on the particle.

### Particle Imaging

Image the particles on the sample using an appropriate technique (e.g., fluorescence microscopy). This image provides a spatial map of all of the particles relative to the sample, and the particle trait of each particle (e.g., green size a).

### Particle Collection

The particles are removed from the surface and collected in bulk.

### Particle Profiling

The bound biomolecule content of each particle is profiled via biomolecule analytical techniques (e.g., sequencing). The unique particle identifier allows the readout from each particle to be identified.

### Assigning Particle Spatial Positions

Similarity between biomolecule profiles of particles is used to assign a spatial position to each particle, based on the particle image via local neighbourhood mapping.

### Example 3

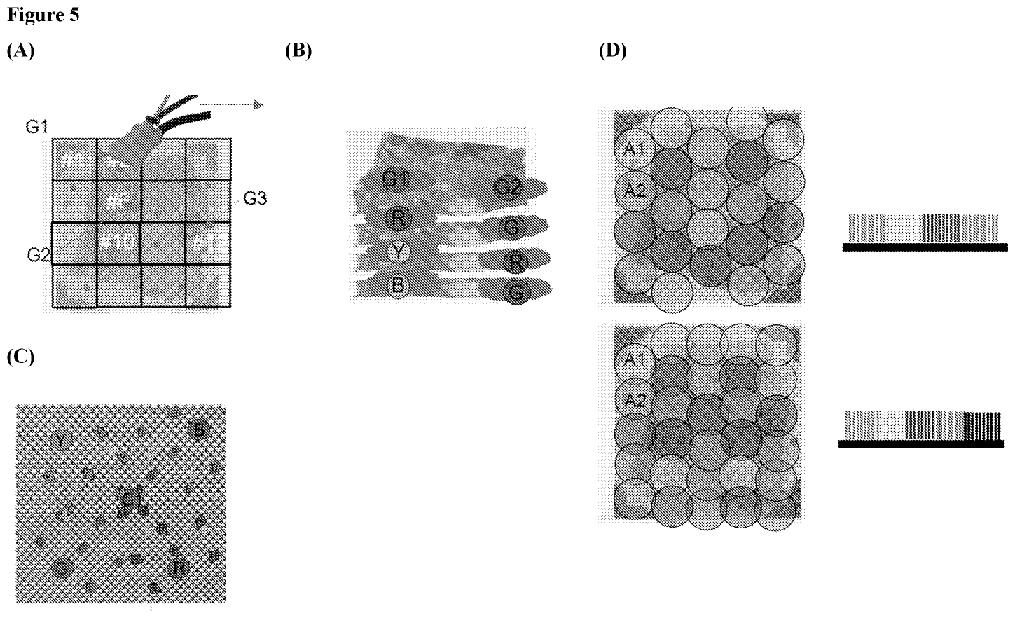

Complementary methods (C1-C4) are described below that would facilitate the core process as described above. These complementary methods are shown in FIG. 5. In particular, to make this system work for larger (up to 1 cm²) samples with denser cell distribution (more than 1 million cells per slice), the read information may need to be complemented by additional information about the spatial origin, which can be provided by the methods below.

### C1: Printed Spatial Barcodes (FIG. 5D)

The method may include the use of spatially patterned surfaces where the spatial information can be transferred onto the particles, implying that back allocation would only be performed in small regions.

Typically, spatial barcodes are printed (but not conjugated) to a substrate (e.g., a glass slide). Alternatively, spatial barcodes may be conjugated to a substrate and possess a cleavable linker (e.g., configured to cleave under thermal or optical stimuli) that may be stimulated to cleave when the sample is in position. Spatial barcodes may be unique DNA sequences. Spatial barcodes are captured by the particles alongside the captured target biomolecules (e.g., RNA, DNA, protein). When the biomolecule content of the particle is profiled, the profile will include the profile of the spatial barcode, allowing the identification of the spatial area in which the particle was originally located. This reduces the computational problem to resolve smaller neighbourhoods.

Barcodes may be printed with no overlap and allowed to diffuse to facilitate measurement of particles which fall on a boundary between two coordinates. Barcodes may be printed to merge intentionally. For example, the particle may contain the biomolecule (e.g., RNA, DNA, protein) AND a DNA barcode which came from A1 OR a DNA barcode which came from A1 and A2.

### C2: Identification Based on Bead Sequence in 3D (FIG. 5B)

The method may be facilitated by taking and analysing multiple slices of the same tissue sample (separated vertically).

A cell may be identified in several slices from the 3D sample based on a similar or identical biomolecule profile of multiple particles (e.g., gene expression pattern). The sequence of particle traits (e.g., bead colours) can be used to assign those particles to a spatial position. For example, as shown in FIG. 5B, the gene expression pattern of G1 appears with GRYB sequence. The gene expression pattern of G2 appears as GGRG. The particle images of the 3D slices can be analysed; a GRYB vertical sequence of particles in the 3D particle image will allow G1 to be assigned the spatial position corresponding to that vertical sequence.

### C3: Artificially Generated/Amplified Neighbourhood Pattern (FIG. 5C)

The method may comprise the release of neighbourhood marker tags from the particles, which will bind to neighbouring particles to create an additional artificial neighbourhood pattern, reinforcing the one inferred solely from the read information.

The marker tags may be optically or thermally triggered to release and bind to other particles, harnessing kinetically limited diffusion so a specific particle only binds marker tags from its neighbours. The marker tag pattern can be read to deduce the neighbourhood. For example, G1 has captured Y, G, B and R marker tags, as inferred during sequencing. Therefore, it can be determined that G1 is in a neighbourhood of YGBR. This can be used to deduce the spatial position of G1.

### C4: Vacuum-Induced Sequential Release (FIG. 5A)

Particles are released from certain portions of the sample at a time. The particles from each portion are profiled separately. This would reduce the number of similarly coloured beads so that reassignment to a spatial position is easier.

### Example 4

To demonstrate how the particle labelling coupled with the neighbourhood information inferred from the resulting data can be used to reconstruct the spatial distribution of the information, the inventors simulated data that mimics what they would get once the full workflow has been realised and performed an example reconstruction on this data. It is to be noted that the example that follows is only one way to perform the reconstruction. Many alternative methods could be possible that all use the neighbourhood information and the labelled particles distributed at density mimicking that of cells.

### Simulating Data

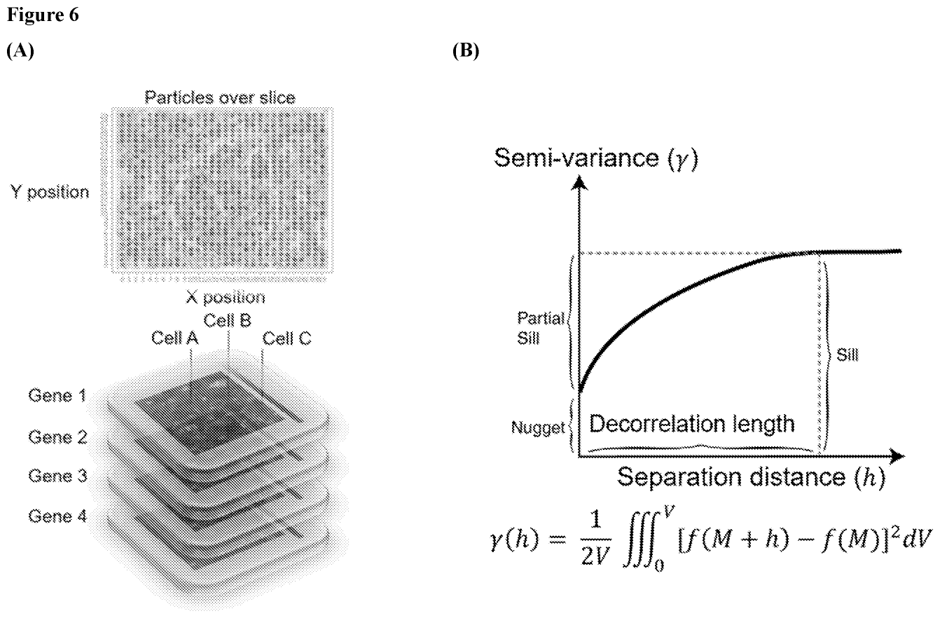

The inventors used Gaussian Random Fields (GRF) to simulate gene expression over a grid of 30×30 size, as shown in FIG. 6A. Gaussian Random Fields are scalar fields where the distribution of numbers is controlled by 3 parameters: the sill, the nugget and the decorrelation length, as shown in FIG. 6B. The sill tells the long distance variance of numbers, the nugget tells the short distance (starting) variance of numbers whereas the decorrelation length defines how quickly the long distance variance is reached. Gaussian Random Fields are used in geography and astronomy simulations where spatial distribution of objects of varying properties are simulated. Gene expression is in principle the same, thus the inventors used GRFs.

The inventors chose a parameter combination (decorrelation length=2, nugget=1, sill=10). They simulated 10 layers for the 30×30 size grid, each of the layers with the same parameters, but due the randomness of the field, yielding a different distribution. In this example, these 10 layers corresponded to 10 different genes this innovation would use for analysis of real world data (which is not available at this resolution and quality).

The inventors also simulated the distribution of particle traits on this 30×30 grid. They chose to simulate 10 different traits and the resulting distribution is shown in FIG. 6A. Here, colours correspond to traits (but the traits may be any suitable trait as discussed in the general description above).

### Embedding Data

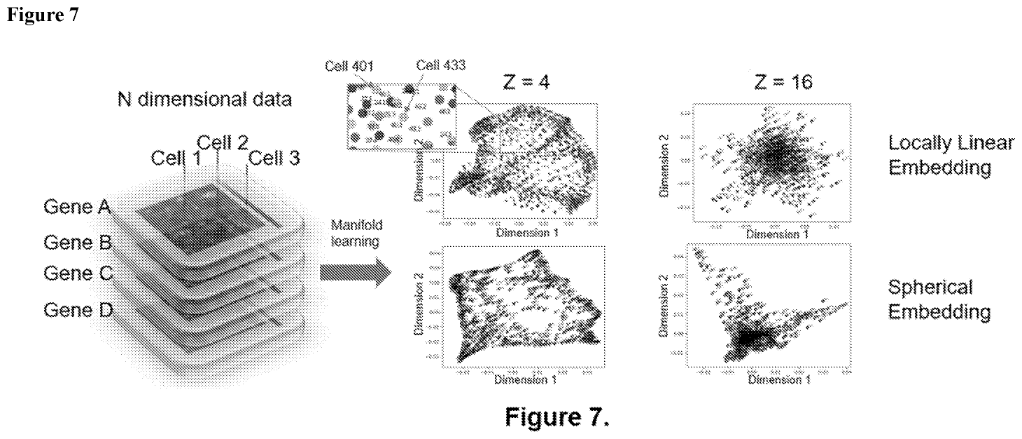

In this example method, the inventors demonstrate one possible way of using the neighbourhood information and the labelled particles to reconstruct the spatial distribution. The first step in this process is the embedding of the 10 dimensional data into two dimensions for later comparison with the physical 2D image in the reconstruction step. Dimensionality reduction (or low dimensional embedding) is a procedure widely used in the machine learning and data analysis community. In this example, the inventors used manifold learning which is a non-linear embedding. This step is shown in FIG. 7. The aim of dimensionality reduction is to map the N dimensional data into 2 dimensional data while maintaining neighbourhood patterns as much as possible.

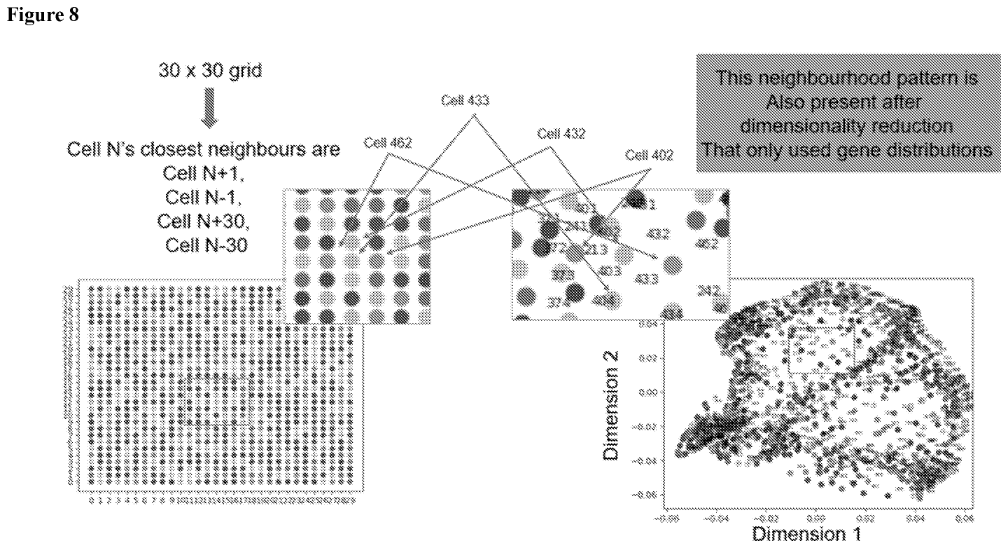

FIG. 8 demonstrates why dimensionality reduction is useful. In a 30×30 grid, cell N's closest neighbours are Cell N+1, Cell N−1, Cell N+30, Cell N−30. After dimensionality reduction, this neighbourhood pattern is preserved.

The manifold learning packages are available for public use. In this example, the inventors used spectral embedding but depending on the quality of data, other types of embeddings or in particular, manifold learning should be used (for example isomaps or locally linear embeddings). One of the merits of manifold learning is that it has a parameter, called neighbourhood size, which allows for the selection of the size of neighbourhood that remains conserved as much as possible during the dimensionality reduction process. Here, the inventors chose a neighbourhood of 4 (Z=4) but this also should depend on the data. Manifold learnings are used in other industries, for marketing and advertisement optimisation purposes where its task is to reveal the relevant information. Similarly, in the current example, it is used to reveal the relevant weighting of the genes to obtain a 2 dimensional ‘gene space map’ from the 10 dimensional data, that is then used in the reconstruction step.

### Reconstructing Data

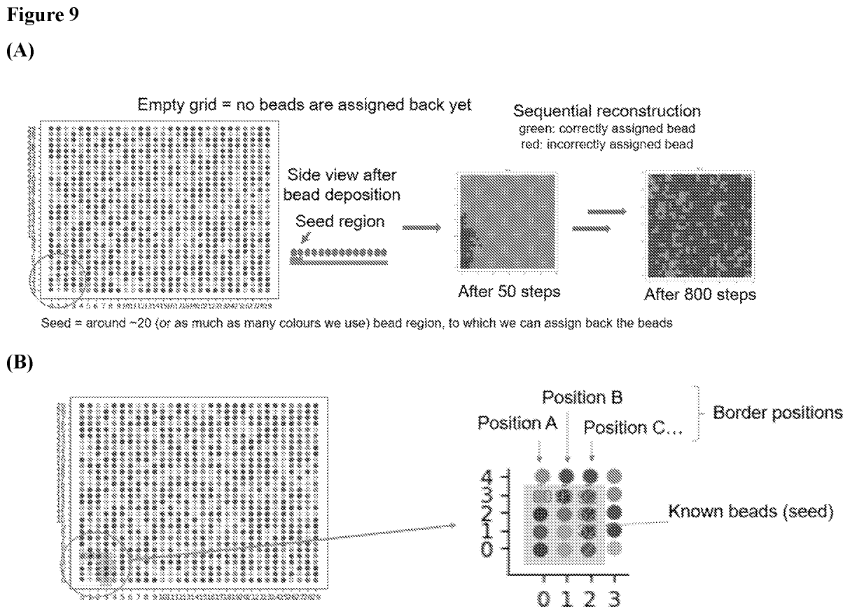

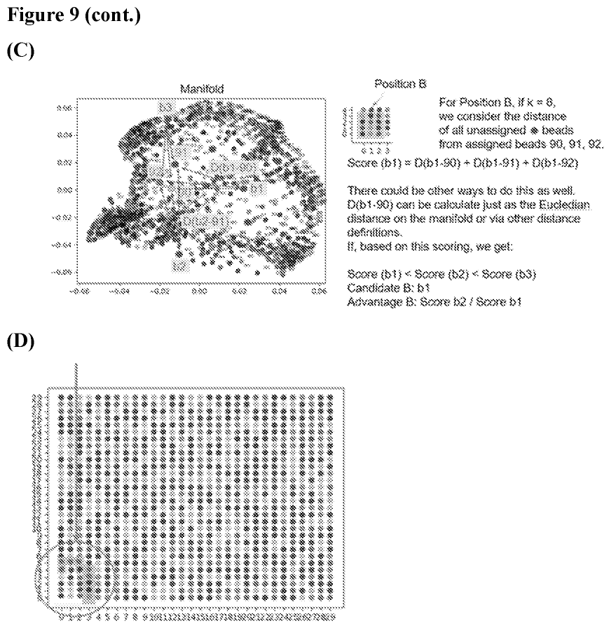

The reconstruction step is the key step in this process. The inventors demonstrate one method here which uses the uniqueness of neighbourhood patterns and the image taken of the labelled particles over the tissue (‘image’). See FIG. 9 for an overview of the steps. The aim of this step is the use the neighbourhood information inferred from manifold learning to assign back as many beads to as many spatial positions as possible.

This method starts with a seed region, as shown in FIG. 9(A). Seed region is a region containing not more particles than the number of different labels (here 10), where the particles can be uniquely assigned back with 100% confidence. A preferred method to achieve this is the printing of ‘seed’ barcodes on a small region on the surface containing the labelled particles, which will bind to and tag those particles at those places. The size of the seed region should be small enough so that statistically only particle per label type binds there allowing back allocation.

The second step is the identification of the ‘border’ region based the image. This is shown in FIG. 9(B). For example, the border position 2 (b2) should have a ‘green’ particle based on the image, whereas b3 a red one. The border region is important because those positions have the most neighbours already assigned, thus it is their reconstruction that can be done with the highest certainty as the next step.

In the next step, for each border position, the unassigned particles are identified (called ‘candidates’). Because each neighbourhood should be unique, in theory one of the candidates should fit better than the others to every border position. To qualify the ‘goodness’ of fit, the inventors evaluated the distance in the ‘gene space’ based on the manifold learned in the previous step, from each of the ‘seed neighbours’ (D) and formed a score as a sum of these (S). Score=sum of distances of the chosen bead to the evaluated positions k nearest neighbours (as inferred from the tissue image) on the 2D manifold. Here, as previously mentioned, ‘neighbours’ refer to the particles that are closer than a certain distance to the border position.

In this example, the distance is taken to be the Euclidean distance on the manifold:

$$D ( A - B ) = \sqrt{{{\sum }_{i = 1}}^{2} {( {dim}_{i} ( a ) - {dim}_{i} ( b ) )}^{2}}$$

which gives the distance between two arbitrary beads A and B. The score of an arbitrary bead, X, is then (as shown in FIG. 9C) calculated as:

$$S ( X ) = {\sum }_{Y = neighbours} D ( X - Y )$$

It is important to emphasize that both the definition of distance and that of score is a representative one here and other definitions are possible.

Once this is calculated for each candidate at a certain border position, the candidates with the two lowest distances are chosen. If the border position had all the neighbours allocated and the embedding was perfect, the true candidate (which is belongs to that border position) would have the lowest distance because it has the unique neighbourhood. However, because by the nature of this method the neighbourhood in most of the cases is not complete and the mapping to lower dimensions is never perfect, instead we use as metric the difference between the two lowest distance candidates distance ratio (A, ‘advantage’). The advantage at position P, where bead X has lowest, and bead Y has second lowest score, is:

$$Ap ( X ) = \frac{S ( X )}{S ( Y )}$$

For example, if green particles A and B, as in FIG. 9C, are the two lowest distance candidates to position b1, the advantage at b1 (belonging to A) is:

$${A}_{b 1} ( A ) = \frac{S ( A )}{S ( B )}$$

Subsequently, the inventors compare the advantages at each of the border positions. They pick the border position where the advantage is the largest. There, they assign the candidate which has the advantage there. For example, green particle A in this example will be assigned to b1 and removed from the pool of unassigned particles, as shown in FIG. 9D. Then in this example method the seed region is updated with the newly assigned particle and the steps of identifying the border, finding the candidates, evaluating the distances and advantages and picking and placing the highest advantage candidate is repeated, until the whole grid is filled.

### Results

The results of this example reconstruction approach can be evaluated by introducing an efficiency metric.

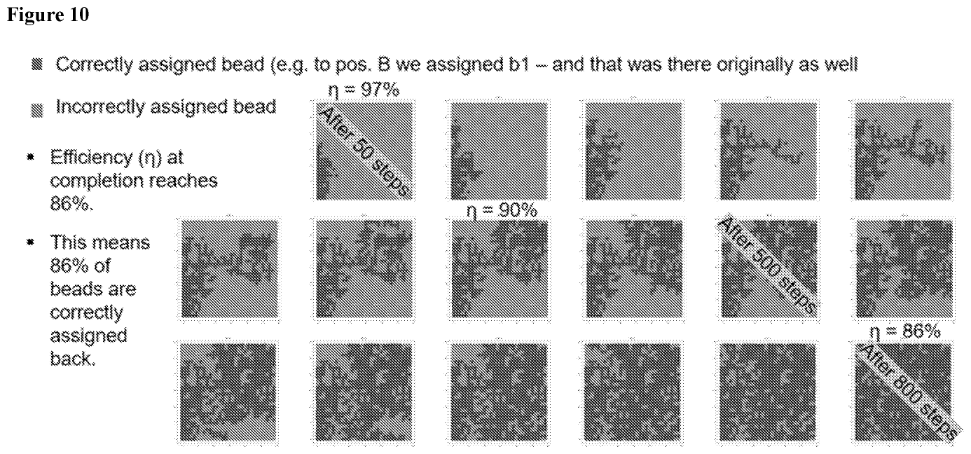

As shown in FIG. 10, the efficiency starts at 100% (the seed region) and declines to reach a quasi steady level around 86% in this example. This suggests that the above method is a viable alternative for the reconstruction. However, it is appreciated that depending on the exact data to be dealt with, the addition of other steps, the change of the parameters value might be necessary. This very versatility is the main advantage of this invention: the key ideas of introducing labelled particles and recognising that the gene space and the real space distance can be used in the reconstruction serve as a basis around which multiple methods can be developed to perform the reconstruction to the highest possible efficiency. This example which uses manifold learning and sequential seeded allocation is one example that works but we anticipate that many, optimised approaches exist.

### Example 5

The inventors further identified that it is not necessary for all of the particles to have a distinguishable trait. Instead, the single cell resolution can also be achieved when a fraction of the particles have a distinguishable trait, and the remaining particles do not have a distinguishable trait. The particles having a distinguishable trait are referred to as ‘landmark beads’. First, the landmark beads are assigned to a spatial position based on the distinguishing trait alone (if each landmark bead has a different trait) or a combination of the distinguishing trait and similarity with other landmark beads. Second, the non-landmark beads are assigned to a spatial position based on the similarity with the landmark beads that are already in position. Accordingly, all of the beads (along with their profiling data) are assigned back to a spatial position such that their profiling data is mapped back to a spatial position of the sample.

### Assigning Landmark Beads to Spatial Positions

To demonstrate how the particle labelling coupled with the neighbourhood information inferred from the resulting data can be used to reconstruct the spatial distribution of the landmark beads, the inventors used published data from a high resolution spatial sequencing technology (Stickels, R. R., Murray, E., Kumar, P. et al. Highly sensitive spatial transcriptomics at near-cellular resolution with Slide-seqV2. Nat Biotechnol 39, 313-319 (2021). https://doi.org/10.1038/s41587-020-0739-1) and manipulated the data to mimic this technology. This dataset includes the exact location and gene expression for each bead. The inventors selected a random sample of the beads to be landmark beads and assigned them to 3 different sub-populations which would correspond to three colours of beads.

It is to be noted that the example that follows is only one way to perform the reconstruction. Many alternative methods could be possible that all use the neighbourhood information and the labelled particles distributed at density mimicking that of cells.

The key assumption is that beads that are neighbours in Physical Space will have bound sequences that are similar, and that the sequence correlation decays reasonably smoothly with distance.
1. This example assumes that out of Nb beads on the slide, a fraction of these beads are optically distinguishable landmark beads (nominally 10%; referred to as ‘Nc’). In this example, the landmark beads are equally divided between the three colours red, green and blue, but the algorithm can handle many more colours (e.g., each landmark bead being a different colour) and other distinguishable traits (e.g., visual barcodes, etc).
2. Simulated data was created as though the slide was imaged to log the X,Y positions of each landmark bead. This provides the data for the Physical Space. It consists of a numbered list of length Nc (of landmark beads) with X,Y cords and a colour.
3. In the dataset used, each bead had a unique identifier, and the genetic sequences were annotated (it is however noted that it is not essential for each bead to have a unique bead identifier if a single cell workflow is used). The data output was the counts of each expressed gene sequence.
4. The raw sequence output was organised into numbered beads (all entries having the same unique identifier were assigned a unique bead number), a sequence name and the number of repeats of that sequence on that bead. This was the data for the Sequence Space, and consisted of a Table of size Ns×Nb with columns corresponding to the assigned bead numbers and rows corresponding to the number of repeats for each sequence for that bead. This was a very large table where the majority of entries were zero (i.e., where a sequence was not present on the bead). Note that the ‘bead numbers’ in the Physical Space do not correspond to the ‘bead numbers’ in the Sequence Space.
5. Summing each column in the Sequence Space results in the total number of sequence expressions on a single bead which is referred to as the ‘Bead Load’. Summing each row results in the total number of expressions of that sequence over all beads, and this is referred to as the ‘Sequence Count’.
6. The landmark beads in the Sequence Space are then separated into a smaller sub-space, having only Nc columns, i.e., Ns×Nc, which is much smaller.
7. The first part of the Algorithm is to establish the physical locations of all the landmark beads in the Sequence Space by using a combination of the position data and the sequences that appear on the beads, i.e., to link the bead numbers in the Physical Space to those in the colour sub-space in the Sequence Space.
8. The inventors generated a table of Physical distances (Euclidean) between the landmark beads in the Physical space. This produced a bead-bead Relative Distance Table of size Nc×Nc where the entries are the distances and the main diagonal was all zeros. Each column of this space was sorted by size ascending, keeping track of the original row number (the original bead number). This was done by introducing a second Nc×Nc Tracked Bead Number Table which corresponded to the distance table, but whose entries are the original numbers of the beads. It was produced by carrying out the same exchanges when the distances were sorted. The colour of each bead was given by its original number. Using the Tracked Bead Number Table, a new Nc×Nc Physical Ranked Colour Table was produced with each entry being the colour of the corresponding bead. The columns in this Table were then the Beads, while the row entries were the colours of the beads at increasing physical distances from the bead of the column. The first row entry in each column was the colour of the bead heading that column. The second row entry was then the colour of its nearest neighbour in Physical Space and so on down the column.
9. A distance metric was devised in the Colour Sequence Space and the same distance-distance/sorting/colour procedure was applied as for the Physical Space. This also produced three Tables: Sorted Relative distance, Tracked Sequence numbers and finally a Sequence Ranked Colour Table. Again, each column represented a bead in Sequence Space where the first entry was the colour of the bead and the second was its nearest neighbour.
10. The first approach was to match all columns from the Physical Ranked Colour Table with all those of the Sequence Ranked Colour Table in pairs, row entry by row entry, and to count the number of hits for each pair of columns. The pair with the greatest number of hits was deemed to have been matched, and their bead numbers could be linked.
11. Step 10 was then repeated on the remaining beads until all the beads are matched.

### Assigning Non-Landmark Beads to Spatial Positions

To demonstrate how the neighbourhood information inferred from the resulting data can be used to reconstruct the spatial distribution of the non-distinguishable beads once the landmark beads arrangement has been reconstructed, the inventors used published data from a high resolution spatial sequencing technology (Stickels, R. R., Murray, E., Kumar, P. et al. Highly sensitive spatial transcriptomics at near-cellular resolution with Slide-seqV2. Nat Biotechnol 39, 313-319 (2021). https://doi.org/10.1038/s41587-020-0739-1) and manipulated the data to mimic this technology. This dataset includes the exact location and gene expression for each bead. The inventors selected a random sample of the beads as the landmark beads for which the known locations were used. The rest of the beads were treated as the non-distinguishable beads—and the given location information not used.

It is to be noted that the example that follows is only one way to perform the reconstruction. Many alternative methods could be possible that all use the neighbourhood information and the labelled particles distributed at density mimicking that of cells.

The process involves assigning each bead back to an estimate of its original position based on its distance from the known positions of the landmark beads.
1. For each bead, the inventors calculated the distance in “gene space” to each of the landmark beads. In this example, the distance is taken to be the Euclidean distance in gene space:

$$D ( A - B ) = \sqrt{{{\sum }_{i = 1}}^{2} {( {dim}_{i} ( a ) - {dim}_{i} ( b ) )}^{2}}$$

which gives the distance between two arbitrary beads A and B in gene space, where the dimensions are the expressions of different genes.
2. The distance in gene space was then used to form probability distributions around each of the landmark beads. The distance was taken as the standard deviation of a 2D gaussian centred on each bead location.
3. The distribution of beads location around each landmark bead was combined by multiplying the 2D probability distributions. The effect of this was to sum the locations of the landmark beads weighted by their distance to the bead.
4. The position estimate for the non-distinguishable bead was taken as the mean of the resulting probability distribution. This was repeated for all of the non-landmark beads.
5. The bead with the smallest standard deviation in its position was then fixed at the nearest non-landmark bead location which can be obtained by either from a bright field image of all the beads or an inferred position by applying a grid of points between the landmark beads using the bead diameter of the pitch.
6. The position estimate for the remaining non-landmark beads can be improved by treating the now fixed position of the first non-landmark bead as a pseudo-landmark bead. For each remaining non-landmark bead, the genetic distance was calculated to each of the landmark beads and the pseudo-landmark bead as in step 1. This was used to generate an additional probability distribution as in step 2 which was multiplied with the probability distribution of the non-landmark bead to update its position.
7. Steps 5 and 6 were then repeated until all the non-landmark beads were assigned a position.

### Example 6

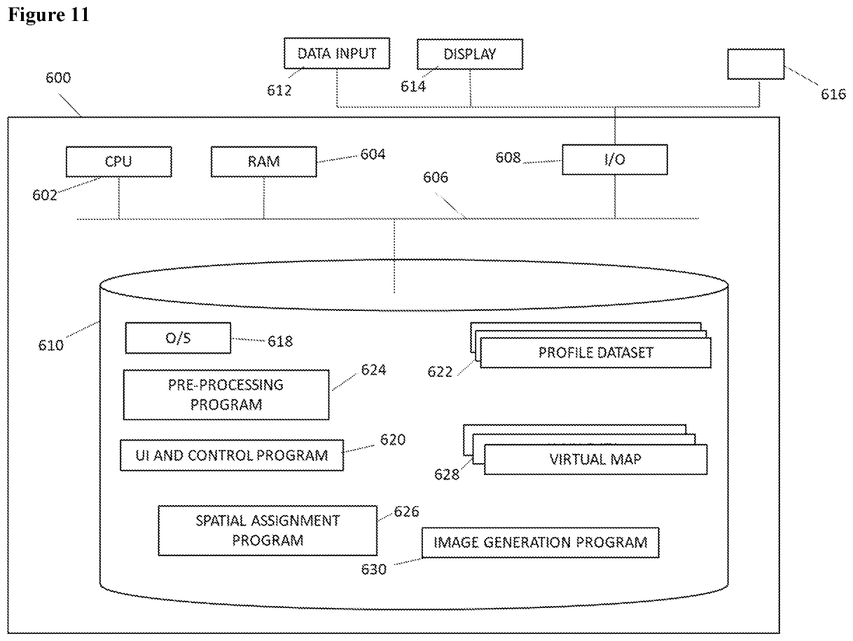

FIG. 11 illustrates an example of a general computing device 600 that may form the platform for various steps of the method of the invention. For example, the computing device 600 may be a mobile phone, a tablet, a wearable computing device, IVI system or the like. The computing device 600 comprises a central processing unit (CPU) 602 and a working memory 604, connected by a common bus 606, and having an input-output (I/O) interface 608 arranged to receive control inputs from a user via a device connected to a data input port 612 such as a keyboard, mouse, touchscreen, push button, or other controller, and provide output information via a user interface which is displayed on a visual display device 614. The I/O interface 608 is also arranged to receive further inputs via various other devices and sensors 616.

The computing device 600 is also provided with a computer readable storage medium 610 such as a hard disk drive (HDD), flash drive, solid state drive, or any other form of general-purpose data storage, upon which stored data, such as a profile dataset 622, and various programs are arranged to control the computing device 600 to operate in accordance with embodiments of the present invention. For example, stored on the computer readable storage medium 610 is an operating system program 618 that when run by the CPU 602 allows the system to operate. Also provided is a pre-processing program 624, a spatial assignment program 626, and an image generation program 630 which together implement steps (h)-(j) of the method according to the present invention when run by the CPU 602, as will be described in more detail below. In order to interface with and control the pre-processing program 624, spatial assignment program 626, and image generation program 630, a user interface and control program 620 is also provided, that controls the computing device 600 to provide a visual output to the display 614, and to receive user inputs via any input means connected to the data input port 612, or any other device connected to the I/O interface 608 in order to control the pre-processing program 624, spatial assignment program 626, and image generation program 630.

Upon receiving instructions to perform a spatial assignment, for example, via the data input port 612, the user interface and control program 620 will extract the relevant data from the profile dataset 622 for input to the pre-processing program 624, which will perform the necessary pre-processing of the source data. The pre-processed source data will then be input to the spatial assignment program 626. The spatial assignment program 616 will then perform the steps of calculating similarity scores for pairs of particles based on the profiling data, and assigning the profiling data corresponding to a particle to a spatial position of a particle in the particle image, based on the similarity scores and/or the particle image, thereby outputting a virtual map 628 of the spatially resolved profiling data with respect to the sample image. This may then be output to the user via the display 614. In this respect, the image generation program 630 may be used to generate a suitable visual representation of the virtual map 628. The virtual map 628 may also be output to the user as a set of raw data, for example, in table format.

Although the invention has been described in relation to one or more preferred embodiments, it will be appreciated that various changes or modification may be made without departing from the scope of the invention as defined in the appended claims.

1. A method of spatially resolved cellular profiling for integrating profiling data of a cell or cell-derived material with spatial positioning of the cell or cell-derived material, the method comprising:
(a) placing a cell or tissue sample onto a sample-receiving surface of a substrate;
(b) contacting the surface of the sample with a population of particles, wherein the particles comprise at least 3 distinguishable subpopulations, wherein each of the at least 3 distinguishable subpopulations has a distinguishable trait that can be determined by imaging, and wherein the particles comprise binding molecules that bind to target biomolecules from the sample;
(c) imaging the sample to provide a sample image;
(d) imaging the population of particles to provide a particle image, wherein the distinguishable trait and spatial positioning of the particles of the at least 3 distinguishable subpopulations can be determined relative the surface of the sample;
(e) capturing target biomolecules from the sample to the binding molecules, such that target biomolecules that are in close proximity to a particle bind to that particle;
(f) removing the population of particles from the surface of the sample;
(g) profiling the particles to generate profiling data corresponding to each particle, wherein the profiling step comprises profiling the target biomolecules bound to the particles;
(h) calculating similarity scores for pairs or pluralities of particles based on the profiling data;
(i) assigning the profiling data corresponding to a particle to a spatial position of a particle in the particle image, based on the similarity scores and/or the particle image; and
(j) providing a virtual map of the spatially resolved profiling data with respect to the sample image.

2. The method of claim 1, wherein
(i) the profiling step comprises determining the sequence of the bound target biomolecules using RNA sequencing, qPCR or mass spectrometry;
(ii) the population of particles is a population of microbeads;
(iii) the target biomolecules are RNA molecules;
(iv) the number of particles in the population of particles is the same as the number of cells within the cell or tissue sample±20%; and/or
(v) the substrate is a microscope slide.

3. (canceled)

4. (canceled)

5. (canceled)

6. (canceled)

7. The method of claim 1, wherein:
(i) the population of particles is fixed to or trapped in a particle-receiving surface of a particle holder substrate, wherein the step of contacting the surface of the sample with the population of particles involves overlaying the sample with the particle-receiving surface of the particle holder substrate; or
(ii) the population of particles is fixed to or trapped in a particle-receiving surface of a particle holder substrate, wherein the step of contacting the surface of the sample with the population of particles involves overlaying the sample with the particle-receiving surface of the particle holder substrate and wherein the particle-receiving surface of the particle holder substrate comprises a plurality of distinct spatial areas, each distinct spatial area comprising a unique spatial identifier tag.

8. (canceled)

9. The method of claim 1, wherein the step of contacting the surface of the sample with the population of particles involves applying a solution comprising the population of particles to the surface of the sample.

10. The method of claim 1, wherein the distinguishable trait is selected from a fluorescent surface label, particle size, particle refractive index, particle shape or a combination thereof.

11. The method of claim 1, wherein the at least 3 distinguishable subpopulations comprises at least 5 distinguishable subpopulations, optionally at least 10 distinguishable subpopulations, optionally at least 20 distinguishable subpopulations, optionally at least 30 distinguishable subpopulations.

12. The method of claim 1, wherein the population of particles comprises fewer than 100 distinguishable subpopulations having a distinguishable trait that can be determined by imaging, optionally wherein the population of particles comprises fewer than 50 distinguishable subpopulations having a distinguishable trait that can be determined by imaging.

13. The method of claim 1, wherein each particle comprises a unique particle identifier tag.

14. The method of claim 1, wherein the particles do not comprise a unique particle identifier tag.

15. The method of claim 1, wherein the steps of imaging the sample and imaging the population of particles are done simultaneously.

16. The method of claim 1, wherein the step of removing the population of particles from the surface of the sample involves removing all of the particles in a single step, or removing particles in sequential steps.

17. The method of claim 1, wherein the sample is a slice of a tissue,
wherein steps (a), (b) and (d)-(g) of the method are repeated on a further slice of the tissue to generate profiling data corresponding to the further slice,
and wherein the calculating step is further based on the profiling data corresponding to the further slice.

18. The method of claim 1, wherein the particles comprise releasable trait identifier tags and trait identifier tag binding molecules that bind to released trait identifier tags, and wherein the method further comprises the steps of:
releasing the releasable trait identifier tags from the particles; and
capturing the released trait identifier tags to the tag binding molecules, such that trait identifier tags that have been released in close proximity to a particle bind to that particle; wherein the assigning step is further based on the captured trait identifier tag profile of each particle.

19. The method of claim 1, wherein the step of calculating a similarity score for each pair of particles involves assessing the similarity of the profile data of a first particle with the profile data of a second particle and assigning a similarity score based on how similar the profile data of the first particle is to the second particle, and repeating for each pair of particles.

20. The method of claim 1, wherein the step of calculating a similarity score for the pairs or pluralities of particles involves calculating the Eucledian distance, the Manhattan distance, the mahalanobis distance, the pearson correlation, the uncentered correlation, the Spellman rank correlation or the absolute or square correlation.

21. The method of claim 1, further comprising the step of applying a similarity score threshold, such that a pair or plurality of particles having a similarity score below the threshold are not considered spatially located within a same neighbourhood, and a pair or plurality of particles having a similarity score above the threshold are considered spatially located within a same neighbourhood.

22. The method of claim 1, wherein the population of particles comprises landmark particles and non-landmark particles, wherein the landmark particles comprise the at least 3 distinguishable subpopulations, and wherein the non-landmark particles do not have a distinguishable trait that can be determined by imaging.

23. The method of claim 22, wherein steps (h) and (i) comprise:
(1) assigning the profiling data corresponding to a landmark particle to a spatial position of a landmark particle in the particle image, based on similarity scores and/or the particle image;
(2) calculating similarity scores for pairs or pluralities of landmark beads with non-landmark particles based on the profiling data;
(3) assigning the profiling data corresponding to a non-landmark particle to a spatial position of a non-landmark particle in the particle image, based on the similarity scores and/or the particle image;
repeating step (3) for other non-landmark particles that have not yet been assigned a spatial position.

24. A particle holder substrate comprising a population of particles,
wherein the population of particles is randomly distributed on a particle-receiving surface of the particle holder substrate,
wherein the particles comprise at least 3 distinguishable subpopulations,
wherein each distinguishable subpopulation has a distinguishable trait that can be determined by imaging, and
wherein the particles comprise binding molecules that bind to target biomolecules.

25. A system comprising:
a processor; and
a computer readable medium storing one or more instruction(s) arranged such that when executed the processor is caused to:
calculate similarity scores for pairs or pluralities of particles based on a set of profiling data;
assign the profiling data corresponding to a particle to a spatial position of a particle in a particle image, based on the similarity scores and/or the particle image; and
provide a virtual map of the spatially resolved profiling data with respect to a sample image.
